(19) United States
(12) Patent Application Publication
(10) Pub. No.: US 2026/0053731 A1
(43) Pub. Date: Feb. 26, 2026

(54) MICRONEEDLE PARTICLES FOR ENHANCED TOPICAL TRANSDERMAL DRUG DELIVERY

(71) Applicants: Theocharis Nikiforos IORDANIDIS, SOLNA (SE); Argyris SPYROU, Norrtälje (SE); Niclas ROXHED, BROMMA (SE); BONSENS AB, LIDINGÖ (SE)

(72) Inventors: Theocharis Nikiforos IORDANIDIS, SOLNA (SE); Argyris SPYROU, Norrtälje (SE); Niclas ROXHED, BROMMA (SE); Göran STEMME, LIDINGÖ (SE)

(21) Appl. No.: 19/260,463

(22) Filed: Jul. 5, 2025

Related U.S. Application Data
(63) Continuation of application No. PCT/EP2024/063237, filed on May 14, 2024.

(30) Foreign Application Priority Data
May 14, 2023 (SE) 23302185

(51) Int. Cl. A61K 9/00; A61K 8/02; A61K 9/14; A61Q 19/00; B33Y 80/00

(52) U.S. Cl. CPC A61K 9/0021; A61K 8/0245; A61K 9/14; A61Q 19/00; A61K 2800/60; A61K 2800/621; B33Y 80/00

(57) ABSTRACT

A microneedle particle arranged to be rolled and massaged on a subject's skin to penetrate the stratum corneum is disclosed. The microparticle comprises a support body, and a plurality of microneedles protruding from the support body (20), each microneedle comprising a tip. The support body maintains the microneedles and tips in a predetermined conformation allowing the microneedle particle to be rolled on the skin. The microneedles have an average tip sharpness radius of less than 20 μm, an average microneedle length of less than 100 μm, and an average tip sharpness to microneedle length ratio of 1:5 to 1:200. Formulations and kits for said use are provided. A microneedle particle manufacturing method using a non-linear two-photon absorption process, a non-therapeutic cosmetic method of skin treatment, and a method of drug delivery through the skin for said microneedle particle are disclosed.

### FIELD OF THE INVENTION

The present invention relates to microneedle particles for enhanced delivery of pharmaceutical and cosmetic compositions to the skin. Also, related manufacturing methods, formulations, and kits for said use are disclosed.

### BACKGROUND

Skin diseases and chronic inflammatory disorders affect a significant part of the global population, with estimates ranging from 30-70% 1-3. They are detrimental physically, psychologically and socially to patients, and their prevalence constitutes a surprising economic burden ¹⁻⁶. Topical transdermal drug delivery would be the preferable method for delivering therapeutics against these conditions ⁷⁻⁹. However, the skin's protective barrier properties, principally the SC layer of the epidermis, have largely prevented the efficacy of liquid and semisolid topical ointments—especially those containing hydrophilic and larger molecular weight compounds ^{1,7-12}.

While systemic administration of therapeutics remains the standard of treatment, it suffers from drawbacks that include toxicity, sub-optimal bioavailability of the compound, and potential long term side effects ^{1,6,13-16}. Despite these drawbacks, many conditions do not allow for the use of oral or intravenous administration of drugs in fear of immune reactions. Moreover, transdermal drug delivery would be preferable to the delivery of drugs that cause irritation/instability in the gastrointestinal tract and/or are deactivated by liver metabolism ^{17,18}. Topical treatment may potentially produce fewer side effects and, in addition, be well tolerated and easier to use for patients compared to oral delivery or injection, especially for groups like the elderly and children.

Such potential advantages have spurred the development of methods to increase the permeability of skin to therapeutic agents previously impossible to deliver in this fashion. Unfortunately, there can be significant negative side-effects if the disruption of the barrier properties of the skin is excessive. Many chemical additives in a formulation can produce irritation ^{7,19} and most mechanical means of disrupting skin barrier properties require auxiliary machinery and devices that introduce complexity, increased cost, and limitations on the area to which they can be applied. The most promising and researched method of these, microneedle array patches (MAPs), shares such drawbacks, mainly the available application area and the need for applicator devices for repeatable performance ^{7,20-27}.

The need to simultaneously satisfy the requirements of a broad-spectrum skin permeability enhancer, easily applied, at low cost, over a large area on the skin by the patient, has resulted in the latest developments in microneedling technology. Microneedle-bearing rollers ²⁸ and particles ²⁹ seek to provide skin barrier disruption over any desired area, without the need for additional auxiliary equipment, electrical or mechanical, or any expertise in administration. The microneedle and device geometry reported for these solutions also presents the opportunity for improvement.

For both rollers and particles, the microneedle size and sharpness can present issues of tolerability and healing of the stratum corneum disruptions. Patient pain or comfort perception is correlated with the length of microneedles, and healing time with the diameter—i.e. the size of the “wound” ^{21,23,30-38}. Ideally the stratum corneum disruption should facilitate the delivery of the agent and then quickly heal to restore the skin barrier properties against infections and not produce any immune response. In the case of additive particles, their geometrical features result in requiring a significant concentration (˜3-10 wt%) of them in a formulation to produce a large number of useful penetrations over the needed area ²⁹. Even though the microneedles are designed to avoid interaction with the skin pain receptors below the epidermis, the pressure required to puncture the skin—a function of needle tip sharpness—can always be felt ^{26,29,38}. Eliminating any discomfort at pore creation would go a long way to achieving tolerability or even a preference from a patient for such methods.

Thus, there is a need for improved transdermal drug delivery techniques in general, and improved devices for delivering pharmaceutical or cosmetic compositions to the skin.

### SUMMARY OF THE INVENTION

It is an object of the present disclosure to provide improved devices for delivering a composition to the skin.

To better address these one or more concerns, microneedle particles for delivery of compositions to the skin, a formulation, a kit, and a manufacturing method having the features defined in the independent claims are provided. Preferable embodiments are defined in the dependent claims.

These, and other objects which are evident to the skilled person from the present disclosure, are met by the different aspects and embodiments of the invention as claimed in the appended claims and as generally disclosed herein.

Hence, according to a first aspect, a microneedle particle arranged to be rolled on a skin (12) of a subject by massaging the particle on the skin of the subject, to thereby penetrate a stratum corneum (14) of the skin is provided. The microparticle comprises a support body (20), and a plurality of microneedles (30) protruding from the support body (20), each microneedle comprising a tip (40). The support body (20) maintains the microneedles (30) and the tips (40) in a predetermined conformation which allows the microneedle particle to be rolled on the skin of the subject. The microneedles have an average tip sharpness radius of less than 20 μm, an average microneedle length of less than 100 μm, and an average tip sharpness to average microneedle length ratio of 1:5 to 1:200.

In one embodiment, said support body may maintain the microneedles and the tips in a predetermined conformation in which the tips of the microneedles define a 3D convex hull configured to be rolled on the skin. Said 3D convex hull may have a shape of a cylindroid, a cylinder, a spheroid, a sphere, a polyhedron, an oloid, a Steinmetz solid, or a Gomboc shape.

The microneedle length may be 10-50 μm, preferably not more than 40 μm. Desirably the microneedle particle may comprise between 10 and 40 microneedles. A distance between two adjacent microneedle tips may be between 10 μm and 100 μm.

The microneedle particle according to the first aspect may comprise a formulation of at least 0.004% wt concentration of microneedle particles.

Desirably, the microneedle particle may comprise a biocompatible material. The microneedle particle may further comprise a biodegradable material. The microneedle particle may be made of a monolithic material.

Preferably, the microneedle particle is made of a material having a Young's modulus of more than 0.5 giga Pascal (GPa).

The microneedle particle may have a largest dimension of up to about 500 μm. Preferably, the microneedle particle may have a largest dimension of more than 40 μm.

In one embodiment, a volume of a single microneedle particle may be from 6.5e-5 mm³ to 2 mm³.

In one embodiment, the microneedles may comprise a coating. Preferably, the coating may be selected from at least one of a ceramic layer, a carbon coating, and an ALD deposition.

In an embodiment, the support body of the microneedle particle may be essentially a sphere.

In one alternative embodiment, the support body of the microneedle particle may be essentially a polyhedron. Preferably, the polyhedron may have equal or more than 4 faces. In a preferred embodiment, the polyhedron may be a tetrahedron, octahedron, icosahedron, dodecahedron, or a dual/compound polyhedral form main body.

In one embodiment, the largest dimension of the support body is 10-500 μm.

The microneedle particle may have a weight of approximately 0.5 to 30 μg.

According to a second aspect, there is provided a formulation comprising a plurality of microneedle particles according to the first aspect and a non-solid medium in which the microneedle particles are dispersed. This formulation is adapted for application to a skin of a mammal.

In one embodiment, a formulation comprises a plurality of microneedle particles (10) arranged to be rolled on a skin (12) of a subject by massaging the microneedle particles on the skin of the subject, to thereby penetrate a stratum corneum (14) of the skin. Each microneedle particle comprises a support body (20), and a plurality of microneedles (30) protruding from the support body (20), each microneedle comprises a tip (40), wherein the support body (20) maintains the microneedles (30) and the tips (40) in a predetermined conformation which allows the microneedle particle to be rolled on the skin of the subject. The concentration of the microneedle particles in the formulation is 0.01% wt or less.

Said non-solid medium may comprise a gel, a serum, a cream, or a lotion. In one embodiment, said non-solid medium may comprise a cosmetic cream or cosmetic fluid. In an alternative embodiment, said non-solid medium may comprise a medicament.

According to a third aspect, a kit is provided comprising a non-solid medium adapted for application to a skin of a mammal, and a plurality of microneedle particles according to the first aspect, configured for subsequent dispersion in the non-solid medium at a time of use.

According to a fourth aspect a non-therapeutic cosmetic method of treatment of a skin is disclosed, comprising the steps of applying the formulation comprising a plurality of microneedle particles or the formulation the skin; applying pressure and massaging/smearing motion to move the microneedle particles on the skin such that the microneedles penetrate the stratum corneum of the skin.

According to a fifth aspect, a method of drug delivery through the skin is provided, comprising the steps of applying the formulation comprising a plurality of microneedle particles according or the formulation to the skin, and applying pressure and horizontal massaging motion to move the microneedle particles on the skin such that the microneedles penetrate the stratum corneum of the skin.

According to a sixth aspect, a method of using a non-linear two-photon absorption process to manufacture a microneedle particle is provided, comprising the steps of a) providing a polymer precursor solution on a substrate and arranging an immersion objective of a 3D printer; b) applying a focused light laser beam through the immersion objective of the 3D printer to the precursor solution to trigger two-photon polymerization (2PP) in a focal spot volume to cure and print a voxel of the polymer precursor solution; c) repeating step b) to move the laser focus along a trajectory in all three dimensions to thereby print the microneedle particles; d) removing uncured precursor solution from the substrate to uncover the microneedle particle; and e) flood UV curing to solidify inner uncured precursor volume in the microneedle particle.

In one embodiment, step a) of the method may further comprise depositing precursor droplets.

In one embodiment, the method may further comprise applying a coating on microneedles. Preferably, the coating may comprise at least one of a ceramic layer, a carbon coating and an ALD deposition.

As used herein, the term the “largest dimension of the microneedle particle” refers to the greatest of the following distances: [1] the distance between the tips of the two microneedles that are the farthest apart (if the microneedle particle includes two or more microneedles), or [2] the farther possible distance between a tip of a microneedle and the side of the core structure that is opposite the side from which the measured microneedle extends.

As used in this description and the appended claims, the singular forms “a,” “an,” and “the” include plural referents unless the content clearly dictates otherwise. Thus, for example, reference to “a microneedle particle” can include a combination of two or more components. The term “about”, as used herein, indicates the value of a given quantity can include quantities ranging within 10% of the stated value, or optionally within 5% of the value, or in some embodiments within 1% of the value.

As used herein, the term “approximately” is to be interpreted as ±5% in relation to a numeric value. Consequently, the term “approximately 100 μm” is to be understood as 95-105 μm. Thus, “approximately 100 μm microneedles” is to be understood as 95-105 μm microneedles.

As used herein, the term “biocompatible” is to be interpreted as a substance that is not harmful of toxic to living tissue.

As used herein, the term “biodegradable” is to be interpreted as a substance capable of being decomposed by bacteria or other living organisms and thus avoiding pollution.

As used herein, the term “largest dimension” of the microneedle particle is to be interpreted as: either the dimension between the tips of the two microneedles that are the farthest apart (if the microneedle particle includes two or more microneedles), or the longest possible dimension between a tip of a microneedle and the side of the core structure that is opposite the side from which the measured microneedle extends.

As used in this application, a “3D convex hull” refers to a convex hull of a set of 3D points; this the smallest 3D convex object that contains all the points in its interior or on its surface.

The term “formulation” is to be understood as a combination of materials and/or substances.

Unless otherwise specified, the term “composition” includes both pharmaceutical and cosmetic compositions. The term composition and compound may be used interchangeably.

The term “medicament” is to be understood a substance or composition that is intended to treat, prevent or diagnose a disease, or to restore, correct or modify physiological functions by exerting a pharmacological, immunological or metabolic action.

The term “pharmaceutical composition” is to be used in its widest sense, encompassing all pharmaceutically applicable compositions containing at least one active substance, and optional carriers, adjuvants, constituents etc. The term “pharmaceutical composition” also encompasses a composition comprising the active substance in the form of derivate or a prodrug, such as a pharmaceutically acceptable salt, sulphate and/or ester of said active substance. The active substance may be any therapeutic or medicinal substance.

The term “cosmetic composition” is to be used in its widest sense, encompassing all cosmetically applicable compositions used for non-therapeutic purposes.

Typically, the subject is a human or animal subject.

In particular, it is noted that, as used in this specification and the appended claims, the singular forms “a”, “an”, and “the” also include plural referents unless the context clearly dictates otherwise.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular embodiment to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1A shows an exemplary microneedle particle: An overview of the function conceptualization and application of rolling microneedle particles (MNP).

FIG. 1B1-1B9 show various embodiments of the 3D convex hull. FIG. 1B1 shows an exemplary 3D convex hull, FIG. 1B2 a cylindroid, FIG. 1B3 a cylinder, FIG. 1B4 a spheroid, FIG. 1B5 a sphere, FIG. 1B6 a polyhedron, FIG. 1B7 an oloid, FIG. 1B8 a Steinmetz solid, and FIG. 1B9 a Gomboc shape.

FIG. 1B10-1B13 show various embodiments of the support body. FIG. 1B10 shows a support body having 4 faces, FIG. 1B11 an icosahedron, FIG. 1B12 a dodecahedron, and FIG. 1B13 a dual polyhedral.

FIG. 1C shows a conceptual illustration of the rolling motion enabled by the MNP geometry and penetration effect on the epidermis. Micro penetrations of the stratum corneum are generated and thus allow compounds to diffuse deeper into the epidermis and dermis, that would otherwise be prevented.

FIG. 1D shows scanning electron micrographs of fabricated MNP, with progressively closer views of a singular MNP and a singular needle. Two-photon polymerization 3D printing can rapidly (<25 s) produce such particles en masse, with repeatable dimensional accuracy. The design geometry and achieved sharpness of the needles provide the ability to penetrate the SC with low force and lower mechanical strength materials, such as methacrylate in this case.

FIG. 1E shows microneedle particles shown on a fingertip for scale in a photograph. Closer optical and fluorescent microscopy views of a singular MNP on a fingertip show the extend of miniaturization.

FIG. 1F shows ex vivo gentian violet staining experiments on porcine skin which reveal the ability of MNP to roll on the SC surface and produce multiple distinct micro penetrations as per design goals. Possible modes of rolling on a surface are expected to produce certain geometric pentagonal patterns. A combination of these motions is observed in the experiments, evidence of the designed rolling motion being achieved during rubbing on skin.

FIGS. 2A-2Q show stratum corneum penetration pattern, pore characteristics and tip sharpness effect on performance. FIGS. 2A-2B show en face images of gentian violet staining of murine skin after manual rubbing (˜40 kPa, 10 sec) (FIG. 2A) without and (FIG. 2B) with the inclusion of 25 MNP. FIGS. 2C-2J show the replication of this experiment in porcine skin produces similar distinct penetrations. (FIG. 2C) Rubbing without MNP, and (FIG. 2D) with 25 MNP, also in progressively higher magnifications (FIGS. 2E-F) to reveal (pore pattern and size plus the lateral diffusion of the dye. (FIGS. 2N-2Q) Cross section imaging of the same porcine skin sample reveal the micro penetration geometry and confirms the damage is contained to SC. Histological staining (FIGS. 2N & 2O) provides information on (FIG. 2N) the SC damage to untreated rubbed skin compared with (FIG. 2O) the inclusion of 25 MNP. (FIGS. PI, 2Q) Cryo-sectioning of the same sample retains the gentian violet dye, showing (FIG. 2Q) the diffusion pattern after penetration compared to (FIG. 2P) SC blocking it when not perforated. FIGS. 2G-2L show microneedle particles with differing tip sharpness were tested on porcine skin. SEM micrographs and corresponding examples of gentian violet staining results are shown for 10 μm tips (FIGS. 2G, 2H), 5 μm tips (FIGS. 2I, 2J), and standard 1μm tips (FIGS. 2K, 2L). FIG. 2M shows perforation count results from these cases are compiled in a graph, for triplicate repeats of application of 25 MNP on a 2 cm×2 cm area of porcine ear skin. Asterisks denote unpaired t-test two tail significance, ns, P>0.05; *P<0.05; **P<0.01; ***P<0.001. Specifically, manual count—10 μm vs 5 μm, P=0.0335, 10 μm vs 1 μm, P=0.0001, 5 μm vs 1 μm, P=0.0013, area stained—10 μm vs 5 μm, P=0.1185, 10 μm vs 1 μm, P=0.0122, 5 μm vs 1 μm, P=0.0112.

FIGS. 3A-3I show ex vivo experimental results of increase in permeability of microneedle-particle-treated porcine skin. Fluorescence of the dermis excluding the stratum corneum (FIG. 3A) was measured in MNP treatment (FIGS. 3B, 3D, 3F, 3H) and negative control cases (FIGS. 3A, 3C, 3E, 3G). Samples were incubated with glycerol gel and fluorescent compound applied topically (FITC-dextran 4 kDa or SRB 0.5 kDa) and cross sections were retrieved at 30-minute and 6-hour time points to assess the permeation of the compound. The compilation of these results is presented in the graph (FIG. 3I). MNP treatment improves the amount of measured fluorescence ˜10× at 30 min (FIGS. 3A, 3B) and ˜7× at 6 h (FIGS. 3C, 3D) for FITC-dextran; and ˜7× at 30 min (FIGS. 3E, 3F) and ˜70× at 6 h (FIGS. 3G, 3H) for SRB. Asterisks denote unpaired t-test two-tail significance, *P<0.05; ** P=<0.01; ***P<0.001. Specifically: FITC-dextran 30 min, P=0.0009, FITC-dextran 6 h, P=0.0034, SRB 30 min, P=0.0009, SRB 6 h, P=0.0006.

FIGS. 4A-4D show that MNP particles boost in vivo doxycycline delivery by shrinking subcutaneous dox-dependent medulloblastoma tumors in mice within four weeks. (FIG. 4A) Photographs of tumor-inoculated mice, analyzed for bioluminescence (BLI) by IVIS on the 0th and 4th week. Mice are imaged by IVIS and pictured bearing tumors of different sizes on their flanks. (FIG. 4B) A dot graph of the individual (for every mouse) BLI signals obtained from the flank tumors at 0- and 4-week time-points are shown. (FIG. 4C) Pie charts give an overview of the survival of mice in topically treated groups after four weeks in the experiment. Non-MNP aided doxycycline delivery does not lead to tumor decrease and survival. (FIG. 4D) A dot-plot shows no significant change in the mouse weight due to MNP treatment among the various groups under the total period of the experiment.

FIGS. 5A-5E show that daily epidermal treatment with MNP and topical doxycycline ointment eradicates dox-dependent flank tumors (Tet-Off) in mice in the course of 9 weeks. (FIG. 5A) Photographs of the tumor-inoculated mice, analyzed for bioluminescence (BLI) by IVIS on the 0th, 5th and 11th week after the start of the treatment. (FIG. 5B) A dot graph of the individual BLI signals obtained from the flank tumors of the epidermally treated groups at 0-, 5-, and 11-week time-points. Dead (x marked) and alive (⋅marked) mice are shown. (FIG. 5C) A dot graph of the individual tumor sizes obtained by Vernier caliper from the flank tumors at 0-, 5-, and 11-week time-points. Only living mice (⋅marked) mice are shown. All 5 mice groups (epidermally and non-epidermally treated) are displayed here. (FIG. 5D) Kaplan-Meier curve for overall survival (11 weeks) of mice bearing flank subcutaneous medulloblastoma tumors. Control mice that received oral dox-water (200 mg/ml) (yellow line) or oral plain water (black line) were also included in the study, revealing that dox treatment will indeed decrease the size of the tumors. Epidermal application included: glycerol with dox (purple line), MNP with glycerol (green line), and MNP with dox (red line). Mice receiving treatment with MNP plus dox (red line) showed a complete recovery under the total period of 77 days, in comparison to the other two experimental groups, MNP+glycerol (green line) and dox+glycerol (purple line), in which the mean survival time were 30 and 35 days, respectively. Log-rank (Mantel-Cox) test and Gehan-Breslow-Wilcoxon test was applied for analysis of the survival curve among groups, 2% dox in glycerol, vs MNP +2% dox in glycerol (**, pMC=0.0017, pGBW=0.0038) , and groups MNP+glycerol vs MNP+2% dox in glycerol (**, pMC=0.0019, pGBW=0.0039). (FIG. 5E) Histological staining reveals no observable differences on the skin of mice after sacrifice for the relevant groups: dox with glycerol, MNP with glycerol, and MNP with dox.

FIG. 6A-6D provide an overview of the MNP fabrication. FIG. 6A shows geometry file (.stl) imported and code generated for controlling the Nanoscribe PPGT2. FIG. 6B provides a schematic overview of the dip-in laser lithography (DiLL) 2-photon-polymerization 3D-printing process for the printing of a single MNP in Nanoscribe PPGT2. FIG. 6C is macro photograph of the substrate after printing with table salt grains for comparison. FIG. 6D provides a SEM micrograph of the MNP array after printing.

FIGS. 7A-7E show SEM micrographs of the different tip sharpness versions for the porcine skin ex vivo penetration experiments. (FIGS. 7A-7C) Closeups of the needles show the difference of 10 μm (FIGS. 7A), 5 μm (FIGS. 7B) and 1 μm (FIG. 7C) tip diameters. (FIG. 7D) An overlayed composite image is provided to aid the comparison. (FIG. 7E) A tilted SEM micrograph of the different versions shows an overview of the geometry of these different MNP-needle length and main body dimensions are identical and only the tip sharpness is varied.

FIGS. 8A-8D: SEM Micrographs of MNP after use on skin. Samples were retrieved after application on skin and placed on carbon tape. No other coating or processing took place before imaging. Needles can either be buckled (FIGS. 8A-8D), blunted (FIG. 8A) or intact (FIG. 8D). There are indications of the punctured corneocytes of the stratum corneum still remaining on some of the needles (FIGS. 8A, 8B, 8D) and/or mucus and dirt (FIG. 8C). The needles that have a flat face due to being the attachment points to the substrate during printing can also penetrate as there is a tip formed on a different orientation (FIGS. 8A, 8B, 8D).

FIG. 9A-9C: Overview of the bioluminescence imaging of tumors in all mice in the animal trial for at the 0-, 5- and 11-week time-points. The mice sacrificed due to tumor burden are marked at the respective timepoint (FIG. 9A). (FIG. 9B) Images of all the mice of the different experimental groups just before their sacrifice. If the sacrifice was due to reaching the maximally allowed tumor burden, the excised tumors are shown. (FIG. 9C) Graph showing the bioluminescence readings for the measurement of the tumor burden on the mice for every week time point.

FIGS. 10A-10D show flowcharts illustrations methods of using a non-linear two-photon absorption process to manufacture a microneedle particle according to the first aspect.

### DETAILED DESCRIPTION

Improved devices, formulations, and kits for delivering compositions to the skin, along with and methods of manufacturing, will now be described in detail.

In embodiments, these devices include microneedle particles that penetrate the top layer of the skin. The top layer of human skin, stratum corneum (SC), blocks the majority of a topically delivered drug and is therefore the most important barrier to cross. The Stratum Corneum is about 10-30 μm thick. In embodiments, the microneedle particles are able to penetrate around 35 μm deep into human skin, allowing a composition to bypass the stratum corneum layer and increasing bioavailability by a very large amount.

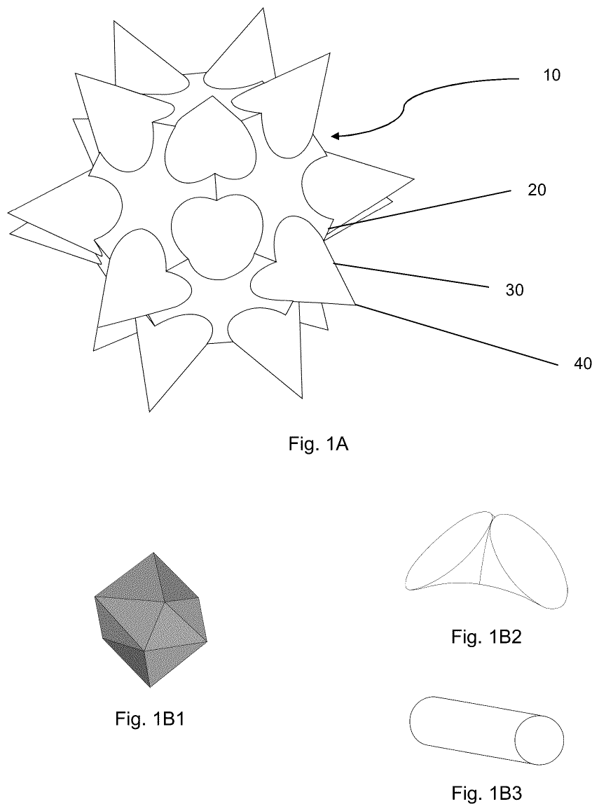

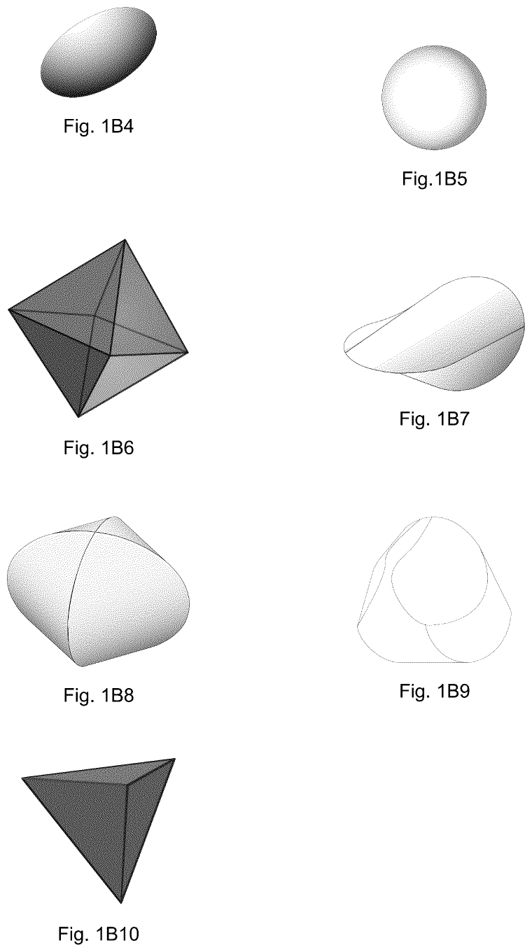

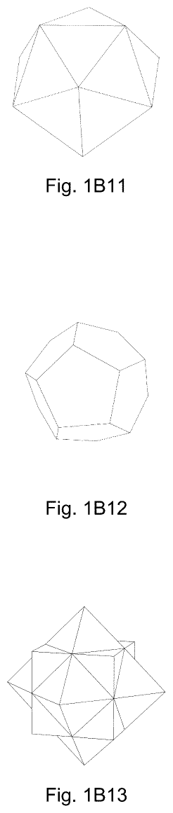

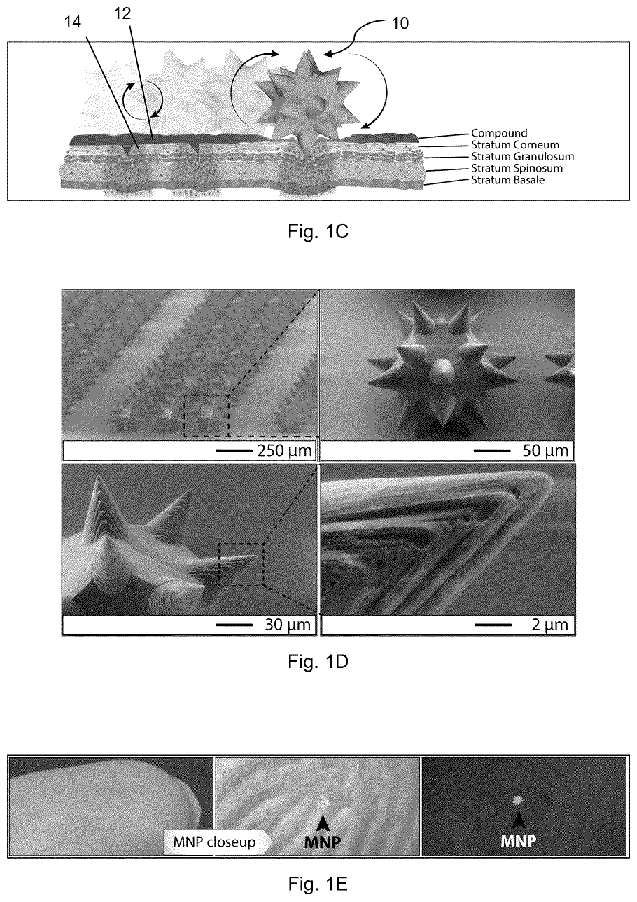

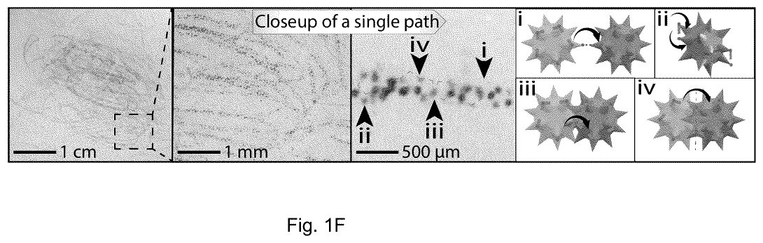

As shown in FIG. 1A, an exemplary microneedle particle (10) comprises a support body (20), from which a plurality of microneedles (30) protrude. Each microneedle (30) comprises a tip (40).

The microneedle particles are designed and structured to roll on skin; when smeared on skin, they create micropores in the stratum corneum. The support body (20) maintains the microneedles (30) and the tips (40) in a predetermined conformation which allows the microneedle particle to be rolled on the skin of the subject. In one embodiment, the support body may be curved.

The microneedles (30) have an average tip (40) sharpness radius of less than 20 μm. In alternative embodiments, the sharpness radius may be approximately, 15 μm, 10 μm, 6 μm, 5 μm, or 1 μm.

The microneedles (30) have an average microneedle length of less than 100 μm, in alternative embodiments, the length of the microneedles may be approximately 50 μm, 30 μm, or 10 μm.

The microneedle particle has an average tip sharpness-to-microneedle length ratio of 1:5 to 1:200. Other embodiments may include for example, 100 μm long microneedles where the tip sharpness may be 1 μm and the ratio of tip sharpness versus length 1:100, 5 μm and the ratio of tip sharpness versus length 1:20, 10 μm and the ratio of tip sharpness versus length 1:10, or 20 μm and the ratio of tip sharpness versus length 1:5. For 50 μm long microneedles, the tip sharpness may be 1 μm and the ratio of tip sharpness versus length 1:50, 5 μm and the ratio of tip sharpness versus length 1:10, or 10 μm and the ratio of tip sharpness versus length 1:5. For 30 μm long microneedles, the tip sharpness may be 1 μm and the ratio of tip sharpness versus length 1:30, 5 μm and the ratio of tip sharpness versus length 1:6, or 6 μm and the ratio of tip sharpness versus length 1:5. For 10 μm long microneedles, the tip sharpness may be 1 μm and the ratio of tip sharpness versus length 1:10, 2 μm and the ratio of tip sharpness versus length 1:5.

The support body (20) may be designed to maintain the microneedles and the tips in a predetermined conformation in which the tips of the microneedles define a 3D convex hull configured to be rolled on the skin. The convex hull configuration may facilitate the rolling of the microneedle particles on the skin. The convex hull may have the shape of a cylindroid, a cylinder, a spheroid, a sphere, a polyhedron, an oloid, a Steinmetz solid, or a Gomboc shape, as shown in FIG. 1B1-1B8.

The microneedle particle may have microneedles with a length of approximately 10-50 μm. Preferably, the microneedles are not more than approximately 40 μm long.

The microneedle particles may have 10 to 40 microneedles; the distance between two adjacent microneedle tips may be between 10 μm and 100 μm. Each one of these parameters facilitates the rolling of the microneedle particles, while creating micropores in the stratum corneum.

The microneedle particle may be dispersed in a formulation comprising at least 0.004% wt concentration of microneedle particles. The formulation may comprise up to approximately 1.7% wt concentration of microneedle particles. For some applications it may be required to have a concentration as low as 0.01% wt or less. Despite this low concentration there exists a distinct technical effect.

The microneedle particle may comprise a biocompatible material, such as a poly-lactice acid (PLA), poly(lactic-co-glycolic acid) (PLGA), polyvinyl alcohol (PVA), polyethylene glycol (PEG), polycaprolactone (PCL), and polyvinylpyrrolidone (PVP), and polymers comprising ethanol or other Class 3 organic solvents (e.g., acetic acid, heptane, acetone, formic acid, isobutyl acetate, etc.). The use of biocompatible materials ensures that the microneedle particles are non-toxic to subjects using them. The microneedle particle may also comprise a biodegradable material that has the advantage of avoiding pollution that might otherwise be caused after its use.

The microneedle particle may be made of a monolithic material. The microneedle material may have a Young's modulus of more than 0.5 giga Pascal (GPa) for allowing penetration of the stratum corneum to create micropores.

The microneedle particle may have a largest dimension of up to 500 μm. In preferred embodiments the largest dimension of the microneedle particle may be approximately 400 μm, 300 μm, 200 μm, 150 μm, 100 μm, 50 μm.

The volume of a single microneedle particle may be between 6.5e-5 mm³ to 2 mm³. In preferred embodiments the volume of a single microneedle particle may be approximately 0.0012 mm³ to 0.06 mm³, more preferably 0.005 mm³ to 0.015 mm³.

The microneedles on the microneedle particle may have a coating. The coating may be a ceramic layer, a carbon coating such as diamond like carbon (DLC), or an atomic layer deposition (ALD).

As previously described, the microneedle particle comprises a support body. The function of the support body is to enable the microneedle particle to roll with ease and create micropores. The support body may be a sphere or a polyhedron. In some embodiments the polyhedron may have four or more faces. In preferred embodiments, the polyhedron may be an icosahedron, dodecahedron, or a dual polyhedral form main body, as shown in FIG. 1B11 and 1B12.

The largest dimension of the support body may be 10-500 μm. In preferred embodiments this largest dimension may be approximately 50, 75, 100, 150, 200, or 300 μm.

The weight of the microneedle particle may be approximately 0.5 to 30 μg.

The microneedle particles may be provided in a formulation to be applied to a skin of a mammal. In this formulation a plurality of microneedle particles may be dispersed in a non-solid medium. The non-solid medium may comprise a gel, a serum, a cream, or a lotion.

In one embodiment, a formulation comprises a plurality of microneedle particles (10) arranged to be rolled on a skin (12) of a subject by massaging the microneedle particles on the skin of the subject, to thereby penetrate a stratum corneum (14) of the skin. Each microneedle particle comprises a support body (20), and a plurality of microneedles (30) protruding from the support body (20), each microneedle comprises a tip (40), wherein the support body (20) maintains the microneedles (30) and the tips (40) in a predetermined conformation which allows the microneedle particle to be rolled on the skin of the subject. The concentration of the microneedle particles in the formulation is 0.01% wt or less.

The non-solid medium may be a cosmetic cream or cosmetic fluid. The cosmetic cream or fluid may comprise at least a cosmetic or cosmeceutical compound. The cosmetic or cosmeceutical compound may be used for longevity or antiaging treatments.

The cosmetic or cosmeceutical compound may be small molecules and large biotechnology produced or purified molecules (e.g., peptides, proteins, DNA, RNA). Examples of biologically active and biologically inactive cosmetic/cosmeceutical compounds, which may include their analogues and antagonists, include, but are not limited to, antiaging products (exfoliants, keratolytic agents, anticellulite agents, antiwrinkle agents, and the like); skin protectants (sunscreens, barrier creams, oils, silicones, insect repellants, itch relief, antiseptics, disinfectants, skin tightening and toning milks and lotions, wart removal compositions, and the like); skin color products (whiteners, lighteners, sunless tanning accelerators, and the like); pigmented skin colorants (face and body makeups, foundation creams, mascara, rouge, lip products, and the like); bath and shower products (body cleansers, body wash, shower gel, liquid soap, soap bars, conditioning liquid bath oil, bath powders, and the like); foot care products, such as keratolytic corn and callous removers, foot soaks, and foot powders (medicated, such as antifungal athlete's foot powder, ointments, sprays, and the like); and antiperspirant powders.

Alternatively, the non-solid medium may comprise a medicament. The medicament may be a pharmaceutical compound or composition. Said pharmaceutical compounds or compositions may include: pharmaceutical ingredients, vaccines, allergens, vitamins, cosmetic agents, cosmeceuticals, diagnostic agents, sensors, markers (colored dyes or radiological dyes or markers), other bioactive agents, and other materials that may be beneficial for penetrating a biological tissue.

In one embodiment, the pharmaceutical compound may be a prophylactic, therapeutic, or diagnostic agent useful in medical or veterinary application. In one embodiment, said compound may be a prophylactic or therapeutic substance, which may be referred to herein as an active pharmaceutical ingredient, i.e., API. In certain embodiments, the API is chosen from among suitable proteins, peptides and fragments thereof, DNA, RNA, and other natural and unnatural nucleic acid-based molecules and fragments thereof, which can be naturally occurring, synthesized or recombinantly produced. Exemplary types of API for delivery include antibiotics, antiviral agents, analgesics, anesthetics, antihistamines, anti-inflammatory agents, anticoagulants, allergens, antineoplastic agents. In certain embodiments, the API may be a dermatological agent used for prophylaxis, therapy, or diagnosis of indications associated with the skin.

In one embodiment, said pharmaceutical compound may comprise a vaccine. Examples of vaccines include vaccines for infectious diseases, therapeutic vaccines for cancers, neurological disorders, allergies, and smoking cessation or other addictions. Also within the scope of the current disclosure are included both current and future vaccines, such as those for the prevention of anthrax, cervical cancer (human papillomavirus), dengue fever, diphtheria, Ebola, hepatitis A, hepatitis B, hepatitis C, Haemophilus influenzae type b (Hib), HIV/AIDS, human papillomavirus (HPV), influenza (seasonal and pandemic), Japanese encephalitis (JE), lyme disease, malaria, measles, meningococcal, monkeypox, mumps, pertussis, pneumococcal, polio, rabies, rotavirus, rubella, shingles (herpes zoster), smallpox, tetanus, typhoid, tuberculosis (TB), varicella (chickenpox), West Nile, and yellow fever.

In another embodiment, said pharmaceutical compound may comprise a therapeutic agent. The therapeutic agent may be selected from small molecules and larger biotechnology produced or purified molecules (e.g., peptides, proteins, DNA, RNA). Examples of therapeutics, which may include their analogues and antagonists, include but are not limited to insulin, insulin-like growth factor, insultropin, parathyroid hormone, pramlintide acetate, growth hormone release hormone, growth hormone release factor, mecasermin, Factor VIII, Factor IX, antithrombin III, protein C, protein S, β-gluco-cerebrosidase, alglucosidase-a, laronidase, idursulphase, galsulphase, agalsidase-β, a-1 proteinase inhibitor, lactase, pancreatic enzymes, adenosine deaminase, pooled immunoglobulins, human albumin, erythropoietin, darbepoetin-a, filgrastim, pegfilgrastim, sargramostim, oprelvekin, human follicle-stimulating hormone, human chorionic gonadotropin, lutropin-a, interferon (alpha, beta, gamma), aldesleukin, alteplase, reteplase, tenecteplase, urokinase, factor Vila, drotrecogin-a, salmon calcitonin, exenatide, octreotide, dibotermin-a, recombinant human bone morphogenic protein 7, histrelin acetate, palifermin, becaplermin, trypsin, nesiritide, botulinum toxin (types A and B), collagenase, human deoxyribonuclease I, hyaluronidase, papain, 1-asparaginase, peg-asparaginase, rasburicase, lepirudin, bivalirudin, streptokinase, anistreplase, bevacizumab, cetuximab, panitumumab, alemtuzumab, rituximab, trastuzumab, abatacept, anakinra, adalimumab, etanercept, infliximab, alefacept, efalizuman, natalizumab, eculizumab, antithymocyte globulin, basiliximab, daclizumab, muromonab-CD3, omalizumab, palivizumab, enfuvirtide, abciximab, pegvisomant, crotalidene polyvalent fab (ovine), digoxin immune serum fab (ovine), ranibizumab, denileukin diftitox, ibritumomab tiuxetan, gemtuzumab ozogamicin, tositumomab, I-tositumomab, antirhesus (rh) immunoglobulin G, desmopressin, vasopressin, deamino [Val4, D-Arg8] arginine vasopressin, somatostatin, somatotropin, bradykinin, bleomycin sulfate, chymopapain, glucagon, epoprostenol, cholecystokinin, oxytocin, corticotropin, prostaglandin, pentigetide, thymosin alpha-1, alpha-1 antitrypsin, fentanyl, lidocaine, epinephrine, sumatriptan, benztropine mesylate, liraglutide, fondaparinux, heparin, hydromorphone, omacetaxine mepesuccinate, pramlintide acetate, thyrotropin-alpha, glycopyrrolate, dihydroergotamine mesylate, Bortezomib, triptoreline pamaote, teduglutide, methylnaltrexone bromide, pasireotide, ondansetron hydrochloride, droperidol, triamcinolone (hex)acetonide, aripiprazole, estradiol valerate, morphine sulfate, olanzapine, methadone hydrochloride, and methotrexate.

In yet another embodiment, said pharmaceutical compound may be a vitamin, herb, or dietary supplement known in the art. Non-limiting examples include 5-HTP (5-hydroxytryptophan), acai berry, acetyl-L-carnitine, activated charcoal, aloe vera, alpha-lipoic acid, apple cider vinegar, arginine, ashitaba, ashwagandha, astaxanthin, barley, bee pollen, beta-alanine, beta-carotene, beta-glucans, biotin, bitter melon, black cherry, black cohosh, black currant, black tea, branched-ahain amino acids, bromelain (bromelin), calcium, camphor, chamomile, chasteberry, chitosan, chlorella, chlorophyll, choline, chondroitin, chromium, cinnamon, citicoline, coconut water, coenzyme Q10, conjugated linoleic acid, cordyceps, cranberry, creatine, D-mannose, damiana, deer velvet, DHEA, DMSO, echinacea, EDTA, elderberry, emu Oil, evening primrose oil, fenugreek, feverfew, folic acid, forskolin, GABA (gamma-aminobutyric acid), gelatin, ginger, Ginkgo biloba, ginseng, glycine, glucosamine, glucosamine sulfate, glutathione, gotu kola, grape seed extract, green coffee, guarana, guggul, gymnema, hawthorn, hibiscus, holy basil, horny goat weed, inulin, iron, krill oil, L-carnitine, L-citrulline, L-trypotophan, lactobacillus, magnesium, magnolia, milk thistle, MSM (methylsulfonylmethane), niacin, olive, omega-3 fatty acids, oolong tea, oregano, passionflower, pectin, phenylalanine, phosphatidylserine, potassium, probiotics, progesterone, quercetin, ribose, red yeast rice, reishi mushroom, resveratrol, rosehip, saffron, SAM-e, saw palmetto, schisandra, sea buckthorn, selenium, senna, slippery elm, St. John's wort, stinging nettle, tea tree oil, theanine, tribulus terrestris, turmeric (curcumin), tyrosine, valerian, vitamin A, vitamin B12, vitamin C, vitamin D, vitamin E, vitamin K, whey protein, witch hazel, xanthan gum, xylitol, yohimbe, and zinc.

In yet another embodiment, pharmaceutical compound may be a therapeutic agent used in dermatology. For example, the therapeutic agent may be small molecules and large biotechnology produced or purified molecules (e.g., peptides, proteins, DNA, RNA), A therapeutic agent for use in dermatology may be a compound used to treat any skin condition, or combination of skin conditions, including bacterial infection, viral infection, fungal infection, alopecia, psoriasis, dermatitis, or photo damaged skin. Antifungal drugs may include, but are not limited to, amorolfine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, or combinations thereof. Antiviral drugs may include, but are not limited to, acyclovir, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, or combinations thereof. Antibacterial drugs may include, but are not limited to, erythromycin, clindamycin, tetracycline, bacitracin, neomycin, mupirocin, polymyxin B, quinolones such as ciproflaxin, or combinations thereof. The therapeutic agents may include immune modulating agents, including, but not limited to, imiquimod. Therapeutic agents for treating photo damaged skin may include, but are not limited to, immune modulating agents or immune activators which are capable of increasing immunity of the human skin mucosa. Non-limiting examples of such drugs include imiquimod, rosiquimod, or combinations thereof. For the dermatological condition of alopecia, the therapeutic agent may include, but is not limited to, corticosteroids, such as betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, minoxidil, spironolactone, finasteride, anthralin, tretinoin topical immunotherapeutic agents such as dinitrochlorobenzene, squaric acid dibutyl ester, diphenylcyclopropenone, other hair growth stimulants, or combinations thereof. For the dermatological condition of psoriasis or dermatitis, the therapeutic agent may include, but is not limited to, corticosteroids, immune modulators, vitamin D3, retinoic acids, or combinations thereof; specific non-limiting examples of such drugs include betamethasone dipropionate, clobetasol propionate, halobetasol propionate, diflorasone diacetate, amcinonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, or combinations thereof.

In a further aspect, a kit is provided that comprises a non-solid medium adapted for application to a skin of a mammal, and, separately, a plurality of microneedle particles; the microparticles are, configured for subsequent dispersion in the non-solid medium at a time of use. Since only a small number of microneedle particles are necessary for obtaining a desired effect and the production of the microneedle particles may be costly, it would be beneficial to apply the non-solid medium and thereafter sprinkle only the needed number of microneedle particles to achieve the desired effect and avoid waste.

In yet another aspect, a non-therapeutic cosmetic method of treatment of a skin is disclosed which involves the steps of first applying the formulation comprising a plurality of microneedle particles to the skin; and then applying pressure and massaging/smearing motion to move the microneedle particles on the skin such that the microneedles penetrate the stratum corneum of the skin.

In a further aspect, a method of drug delivery through the skin is provided which comprises applying the formulation comprising a plurality of microneedle particles to the skin and then applying pressure and horizontal/massaging motion to move the microneedle particles on the skin such that the microneedles penetrate the stratum corneum of the skin.

The microneedle particles are manufactured using a non-linear two-photon absorption process. A method of using a non-linear two-photon absorption process to manufacture a microneedle particle comprises the steps of a.) providing a polymer precursor solution on a substrate and arranging an immersion objective of a 3D printer; b.) applying a focused light laser beam through the immersion objective of the 3D printer to the precursor solution to trigger two-photon polymerization (2PP) in a focal spot volume to cure and print a voxel of the polymer precursor solution; c.) repeating step b) to move the laser focus along a trajectory in all three dimensions to thereby print the microneedle particles; d.) removing uncured precursor solution from the substrate to uncover the microneedle particle; and e.) flood UV curing to solidify inner uncured precursor volume in the microneedle particle.

In one embodiment of this method, step a) may include depositing precursor droplets. The depositing may include squeezing out or expressing the precursor droplets.

The method of manufacturing may further comprise applying a coating on microneedles. In certain embodiments, the coating may be a ceramic layer, a carbon coating, an ALD deposition, or a combination thereof.

### EXAMPLE 1

### Clinical Study to Design, Fabricate, and Test Microneedle Particles to Increase Delivery of Compounds to the Epidermis

### Materials and Methods, Study Design

The aim of this study was to design, fabricate, and test engineered microneedle particles (MNP) which generate painless micro-penetrations of the SC. The goal of this technology was to increase the delivery of compounds through the epidermis, to establish the importance of ultraminiaturized, ultrasharp microneedle spheres as topical drug delivery facilitators. The ability of the MNP to perform micro penetrations on the skin models and increase the diffusion of fluorescent markers into the skin was characterized. For this, murine skin from the flank of mice and porcine skin from the ears of pigs with specific numbers of samples and experimental replicates was used, as indicated in the figures and main text. No randomization, or prior power calculations were performed for in vitro experiments. Randomization, and prior power calculations were performed for in vivo experiments. Calculations of a desired sample size has been performed to verify the number of animals needed for these experiments. For determining the sample size in this animal study, performing the resource equation approach applying ANOVA calculations (E-value), it was concluded that having five groups (three experimental and two controls) and five animals per group would be ideal for performing statistically significant experiments.

Replicates varied depending on the experiment. All in vitro experiments were done in technical triplicates, unless otherwise mentioned. Assessment of the MNP efficiency on porcine and murine skin required excision of ears from pigs and skin from flank of mice. This was followed by treatment with MNP, GV-staining, manual counting of the micro penetrations and calculating the percentage of the covered area by the GV-stain after treatment (automatic by ImageJ). Thus, this procedure involved taking four representative images per MNP application (three repeats in total) per condition from three biological repeats (n=9). Evaluation of the FITC-dextran and SRB diffusion into excised porcine skin was done after 30 min and 6 h under fluorescence microscopy by taking five to ten representative slices per condition (n=5).

The in vivo trials were performed using mice under daily topical skin treatment with established flank tumors. Subcutaneous tumors were induced in mice by injecting a transgenic Tet-Off regulated doxycycline-dependent murine cancer cell line into the flanks. The rationale behind the selection of a Tet-Off tumor model was based on the capacity to turn on or off an oncogene (MYCN) which will directly translate into the growth or shrinking of the tumor, respectively. All groups had similar average weight across all groups. Mice were grouped in such a way that the tumors were in similar size across all the groups at the start of the treatment. Conditions were assigned randomly to these groups. This study was not blinded.

### Design and Manufacturing of Microneedle Particles

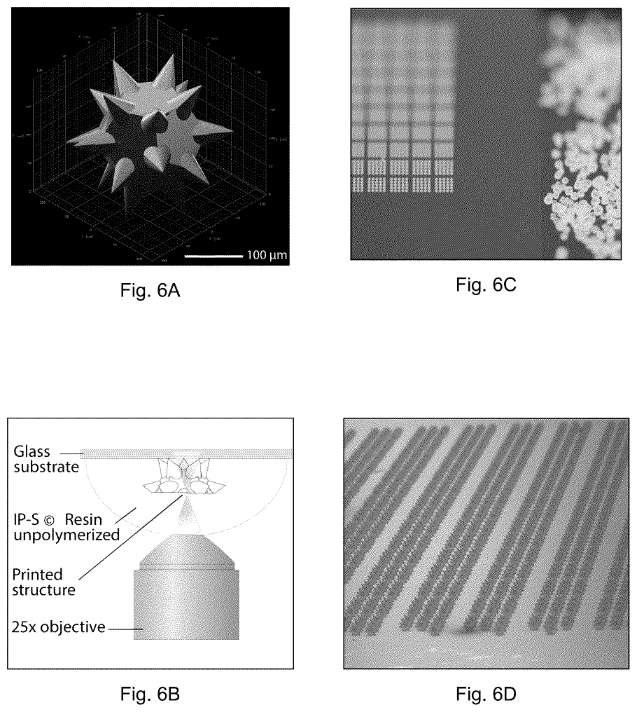

Microneedle particles were manufactured using a 2-photon polymerization method. Specifically, a Nanoscribe© PPGT2 (Nanoscribe GmbH—BICO group, Karlsruhe, Germany) printing tool and the 3D microfabrication solution set for medium features (3D MF) were used. The Nanoscribe© PPGT2 refers to the tool configuration with a Carl Zeis© 25×/NA0.8 immersion objective for focusing the laser beam and the non-cytotoxic, epoxy-based IP-S© resin for printing structures on an indium-titanium oxide (ITO) coated fused silica substrate. The resin in its liquid form were placed on the substrate and then placed above the objective for printing in a dip-in laser lithography mode. FIG. 6B provides a schematic overview of the dip-in laser lithography (DiLL) 2-photon-polymerization 3D-printing process for the printing of a single MNP in Nanoscribe PPGT2.

Briefly, the objective touched the resin and focused through it on the substrate. A near-infrared laser beam coming from the source and reflected off a galvo mirror system was focused through the objective and, utilizing a nonlinear photon absorption effect, enough light intensity was generated to exceed the polymerization threshold of the otherwise near ultraviolet sensitive resin. The absorption probability pertaining to this effect resulted in a very small focal zone and therefore to the excellent resolution of this method. The voxel size that could be printed was a function of the resin properties, the objective optical properties and the laser beam power and scanning speed, set as printing parameters in the printing recipe. The desired beam was scanned over a defined path by the movement of the galvo mirror in the xy plane, and the substrate moved utilizing a piezo motor stage in the z axis after each defined layer, to produce the 3D structure. With these parameters, microneedle particles were fabricated with an overall size smaller than the available printing field (Ø480 μm) of this objective, with the current examples being approximately 235 μm. The microneedle particles had a main body in the shape of a regular dodecahedron, with 40 μm long penetrators of ≈1 μm tip radius based on each of its vertices. Printing time was ˜30 seconds/particle. A typical fabrication workflow consisted of designing the desired SMS shape in a CAD software (e.g. SolidWorks©), exporting the .stl geometry file to the DeScribe© tool software where slicing and translation to g-code took place, and then loading the printing job file onto the tool and NanoWrite© software, so that the structures could be printed on the substrate as mentioned above. To improve throughput, the printing parameters were optimized with respect to the geometry slicing, laser power and scan speed, printing the geometry as shell contours and leaving the inner volume uncured, and of course the way an array of thousands of such particles was printed on each substrate (25 mm×25 mm). After printing, the substrate with the structures underwent a development routine to remove uncured resin and then flood UV curing to solidify the inner uncured volume. The particles were then stored “as-is” on the substrate and ready to use; they were easily removed from the substrate surface to be placed directly onto the application area or into a cream.

### Scanning Electron Microscopy of MNP

MNP were inspected using scanning electron microscopy (SEM) (Zeiss Ultra 55, Munich, Germany, Hitachi S-3400N, Hitachinaka, Japan and Helios 5 UC FIB/SEM, Hillsboro, OR, USA) and pictures were taken on different magnifications for structure analysis and observations. The samples were not coated with other conductive materials.

### Experimental Preparation of Porcine and Murine Skin

Freshly excised porcine skin from the abdomen and ears was obtained from Karolinska Experimental Research and Imaging Centre (KERIC). After excision, the skin tissue was washed at room temperature with lukewarm tap water. An area of 50 cm² skin was prepared for every trial. Regarding murine skin, hairless nude mice were euthanized and an area of 9 cm² skin was excised from the flanks for experimentation. Both types of skin were then dissected from any cartilage or unwanted fat tissue and scissors were then used to clear excess hair without disrupting the upper skin layers. The skin was immediately used for the experiment or stored at −20° C. until further use.

### Application of MNP on Porcine and Murine Skin Ex Vivo

MNP were used on dry skin to assess their utilization in murine and porcine skin. Each trial included immobilizing the skin on a dry and flat surface under modest tension by using paper pins. The MNP were carefully detached from the coverslip that they were printed on by using thin forceps. 25 MNP were placed on the skin for every application to evaluate the number of penetrations that were generated. The MNP were then tenderly rubbed onto the skin with a gloved index finger using moderate pressure (˜40 kPa) in a circular motion for 10 s. After their application, the MNP were gently cleaned off by wiping with wet wipes.

### Histological Preparation and Staining of Skin

For hematoxylin/eosin staining (H&E), skin was isolated, immediately fixed in PFA (4% paraformaldehyde in PBS) overnight at room temperature, and then transferred to 70% ethanol prior to paraffin embedding and sectioning. Then, skin sections (8 μm) were stained with hematoxylin for 3 min after rehydration, rinsed for 10 min in running tap water and then stained for 1 min in eosin solution before being mounted on Fluoromount (DAKO). Whole slide pictures were taken by Olympus Slide View VS200 at Histological Core facility—HistoCore, Karolinska Institutet, Sweden, processed, and analyzed by OlyVIA software.

### Ex Vivo Assessment of Skin Permeability Post MNP Application

Treated and control porcine skin was used to evaluate skin permeability by 4 kDa Fluorescein isothiocyanate (FITC)-dextran (#FD4, Sigma-Aldrich, Stockholm, Sweden) and 0.56 kDa Sulforhodamine B (SRB) (Cat nr #230162, Sigma-Aldrich, Stockholm, Sweden) fluorescent dyes. The skin tissues were placed on the top of 6 cm cell culture dishes filled to the top with 1×PBS with the dermis layer facing downwards and being submerged in PBS, while the epidermis facing upwards for being available to our treatment. Skin, which was stored, refrigerated, or frozen was put in PBS prior to letting it rehydrate at 4° C. for approximately 6 h. FITC-dextran (10 μM) and SRB (10 μM) dyes were mixed in glycerol (EAN: 07350062160239, Frostpharma AB, Danderyd, Sweden) and were added on the epidermis which then was covered with parafilm to avoid evaporation. The set up was carefully monitored throughout the experiment and reagents were carefully added in case of evaporation. Samples were taken out in 30 min and 6 h after the addition of the dyes to evaluate permeability. The skin samples were then washed thoroughly with PBS, dried on dry napkins, and were subjected to cryo-sectioning. The skin sections were then visualized by using Zeiss AXIO Scope A1 Microscope with Axiocam 105 color (Munich, Germany) and quantified with ImageJ v1.53.

### Ex Vivo Gentian Violet Staining of the Skin and Visualization of Penetrations

Skin treated with MNP was submerged in gentian violet (Cat. nr #48770, Sigma-Aldrich, Stockholm, Sweden) 1% w/v in methanol/water for 20 min to expose regions of the stratum corneum that were penetrated by the MNP. The skin was then removed from the gentian violet solution and washed with water and 70% ethanol until all the excess and nonadherent dye was washed away. The skin was then dried up in dry napkins and processed for visualization. Images of the skin samples were visualized by Zeiss Axioscope 5 with Axiocam 208 color (Munich, Germany) and Leica M205C Stereo Microscope (Wetzlar, Germany) and processed in ImageJ v1.53 to analyze gentian violet staining. Skin penetrations were visible as blue stained dots at the sites of penetrations, which were manually counted to avoid any software errors.

### Cell Culture

The GTML2 cell line model used in this study has been previously described ⁴⁰. Tumor cells were cultured in neurobasal media without vitamin A (Cat nr #10888022, Gibco, Stockholm, Sweden), supplemented with antibiotics Penicillin-Streptomycin (10,000 U/mL, Cat nr #15140122, Gibco), B-27 Supplement minus vitamin A (Cat nr #12587010, Gibco), L-Glutamine (Cat nr #25030081, Gibco) and 20 ng/ml EGF (Cat nr #E9644, Sigma, Stockholm, Sweden), and 20 ng/ml FGF-2 (Cat nr #100-18B, Peprotech, Rocky Hill, NJ). Cells were cultured and propagated on ultra-low attachment flasks (Cat nr #CLS3814, Corning, Stockholm, Sweden) until cell spheres were formed (80-90 % confluency), passaged, and used for the in vivo experiments.

### Animal Studies

Hairless NMRI Nude mice (6-8 weeks old of homozygous Nu/Nu) were purchased from Charles-River (Germany). All mouse experiments were performed at KM facilities of Karolinska Institute (KI) in accordance with Swedish legislation and approved by the Stockholm's Ethics Committee for the use of laboratory animals. All animal studies comply with the National Committee for the Protection of Animals used for Scientific Purposes' recommendations and were carried out in accordance with the EU Directive 2010/63/EU associated guidelines under the ethics permit for animal experimentation with number 2332-2023 approved by the Swedish Agricultural Agency.

### Injection of Tumor Cells in the Flanks of Mice

GTML2 cell spheres were gently dissociated into single cells, counted, and single cell suspension (6*106) was injected in the flanks of hairless NMRI Nude mice (˜20 g, 8 w at the start of the experiment) in a volume of 100 μl per flank. The mice were monitored for signs of discomfort (hunched posture, immobility, >10% weight loss) throughout the duration of the experiment as per ethics application.

### Epidermal Treatment of Flank Tumors

In vivo experimentation was performed by inducing subcutaneous GTML2 tumors in mice through injecting a Tet-Off transgenic doxycycline-dependent murine cell line 40 into the flanks. In the absence of dox, the cells from a bidirectional Glutamate transporter 1 (Glt1) promoter expressed both Luciferase (Luc) and the MYCN gene, the latter which was required for their growth and proliferation. In the presence of dox, Luc and the MYCN gene became inactivated, consequently the cells failed to survive, and the tumors diminished. Mice were monitored daily and were visualized with IVIS under tumor suspicion. Once the flank tumors reached around ˜150-250 mm³ (palpable) in size, the mice were randomized into five groups of three-five and the treatment started. The mice were divided into five groups: Firstly, two control groups not involved in skin treatments; (i) one negative control group receiving doxycycline (200 μg/ml) in drinking water (water dox (−)), and (ii) one positive control group receiving plain clean water in drinking water (normal water (+)). Epidermal treatment groups: (iii) one group receiving treatment of MNP with glycerol on the skin (MNP+glycerol), (iv) one group receiving treatment with 2% doxycycline in glycerol on the skin (2% dox in glycerol), and (v) one group receiving treatment with MNP with 2% doxycycline in glycerol on the skin (MNP+2% dox in glycerol). All the mice always had access to clean drinking water, except the ones that had always access to drinking water complemented with doxycycline (water dox (−)). The epidermal treatment groups (iii), (iv), and (v) received daily skin treatment for the total period of 11 weeks. Approximately ˜50 MNP/flank/mouse were placed carefully on the skin of the right flanks of mice above the developed tumor. The MNP then were gently rubbed in circular motion at a pressure of ˜40 kPa for 10 s on around 2 cm² of skin followed by application of; glycerol, or 2% doxycycline in glycerol ointment for 30 min. Animals were treated for 76 consecutive days (11 weeks) according to the initial plan and as per ethics application. Tumors were monitored and measured by IVIS imaging and by vernier calliper every week until the end of the experimentation. When measuring using a Vernier calliper, tumor volumes were calculated from the ellipsoid formula: V=4/3 πWLD. Mice were sacrificed when tumors reached a volume of 1.5 cm³. Skin tissues and flank tumors were collected after the mice were sacrificed for further analysis.

### In Vivo Imaging of Flank Tumors

For in vivo imaging, five mice per group were visualized at a time. Mice were anesthetized by isoflurane, injected intraperitoneally with D-luciferin (150 mg/kg body weight) (Cat nr #ab145164, Abcam, Cambridge, UK) and were incubated 10 min until visualization. The intensity of the bioluminescence signal (BLI) expressed as photons per second (p/s; Total flux) was captured using an IVIS SpectrumCT preclinical in vivo imaging system (PerkinElmer, Waltham, Massachusetts, USA) and analyzed by Living Image v4.7.3 software. To further visualize and quantify the luminescence intensities, Aura Imaging Software v4.0.8 was used and a specific region of interest (ROI) was selected in all the animals at all weeks.

### Statistical Analysis

Statistical analysis was performed using GraphPad Prism version 8.0 (GraphPad Software, Inc.) or Microsoft Excel. Survival curves of the mice were plotted by using the Kaplan-Meier settings, using the GraphPad Prism 8.0 software. The data presented were means ±SD, unless otherwise specified in the figure legends. Student unpaired t-test was used to compare groups for single and multiple comparisons.

### Results

3D Polymeric Microneedle Spheres have Ultrasharp Needles

To achieve truly microscale features that enabled the microneedle particles to perform in a safe and imperceptible manner, novel fabrication methods had to be employed. Two-photon polymerization (2PP) provided the necessary voxel size resolution for the printing of micron tip diameter needles (FIG. 1D, 6A-D, 7A-D)³⁹. Such sharpness was necessary for the needle's function, especially if the printable polymeric materials were considered. Commercially available tools, in this work the Nanoscribe PPGT2, provided this sharpness along with the ability to print complex 3D designs with overhanging parts, at an appreciable time scale. To achieve the scalability of production needed for the translation of this technology, the printing speed had to be maximized. The inventors' optimization of the geometrical design and printing parameters resulted in a 3D microneedle sphere that could be printed in ˜25 s with our current tool. SEM imaging of the more than 2500 MNP instances per printing run revealed the excellent repeatability of the production method, with all the structures exhibiting the same sharpness (FIG. 1D). Barring printing tool malfunctions, the yield of a printing process approached 100%.

### Rolling Motion of MNP Multi-Porated Porcine and Murine Skin Ex Vivo

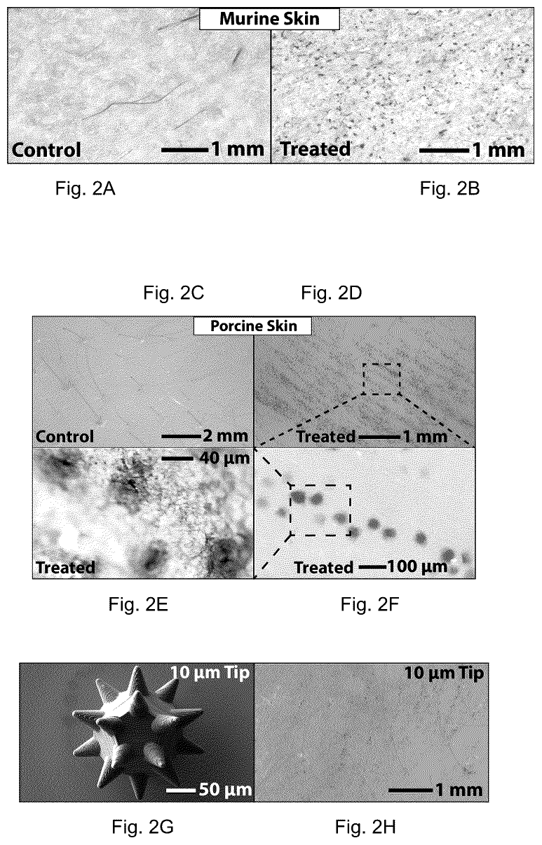

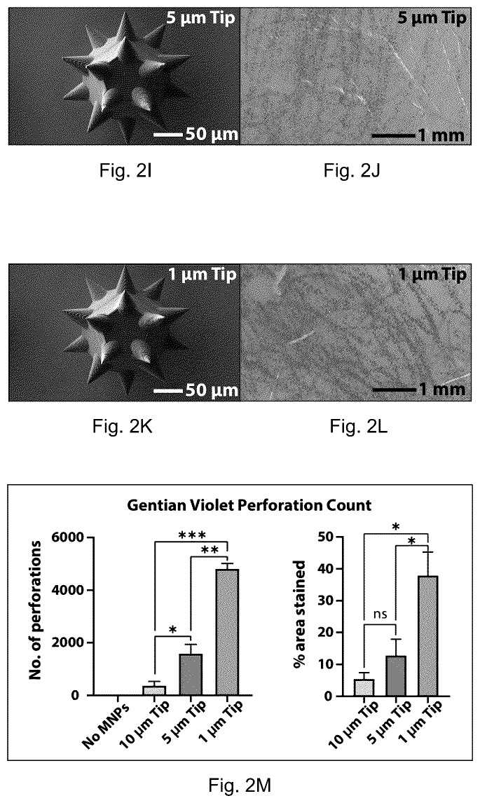

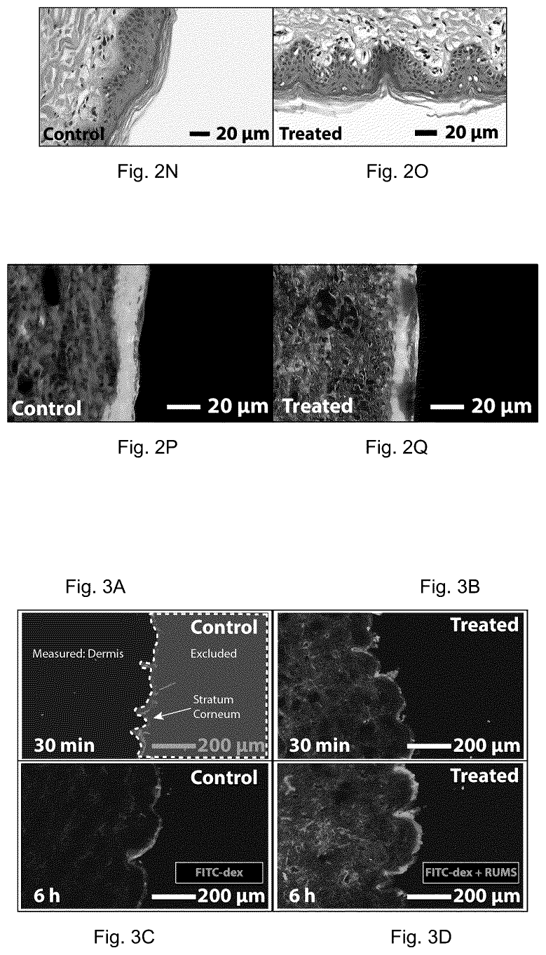

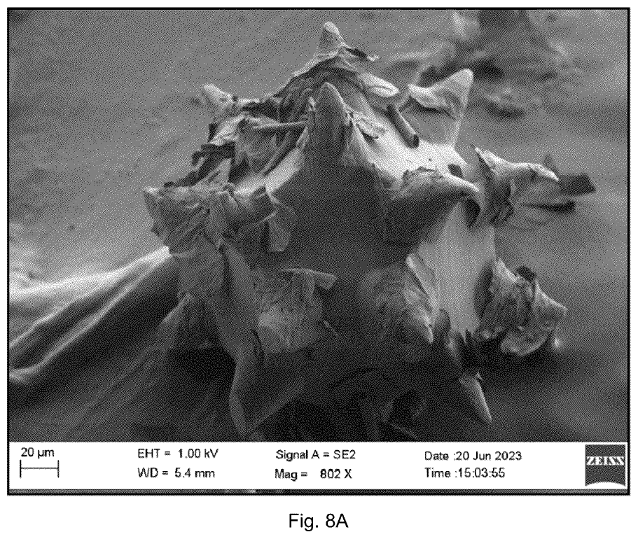

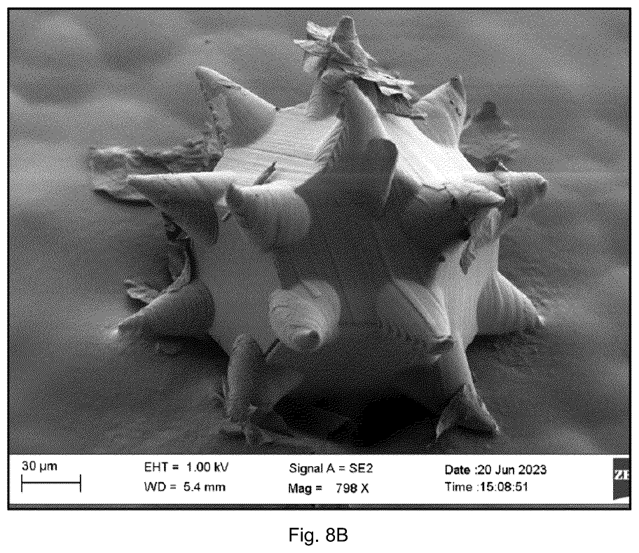

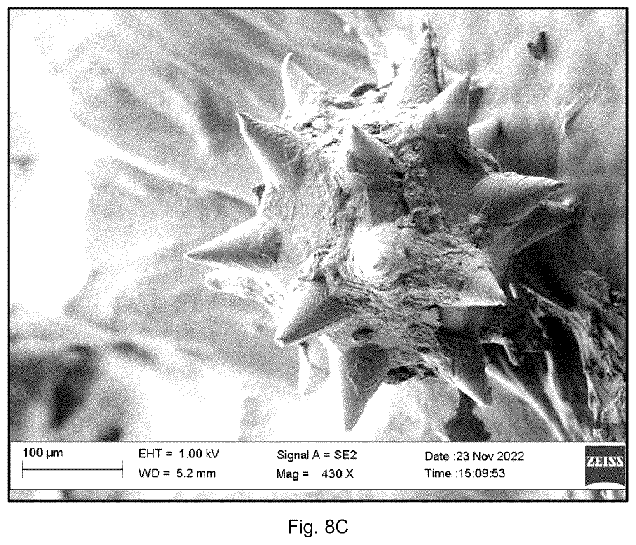

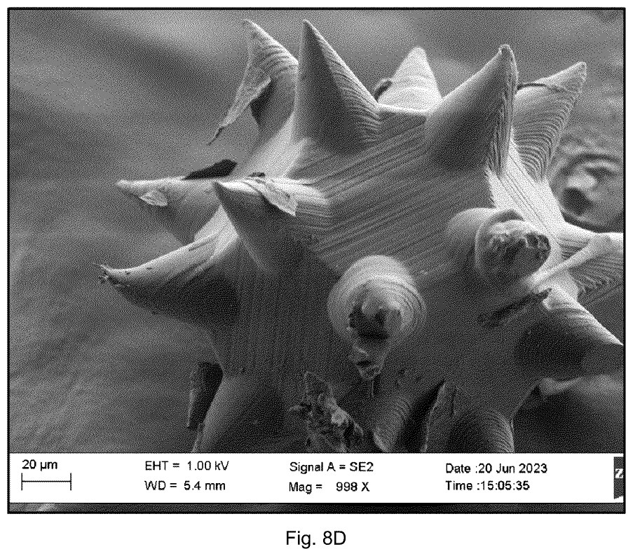

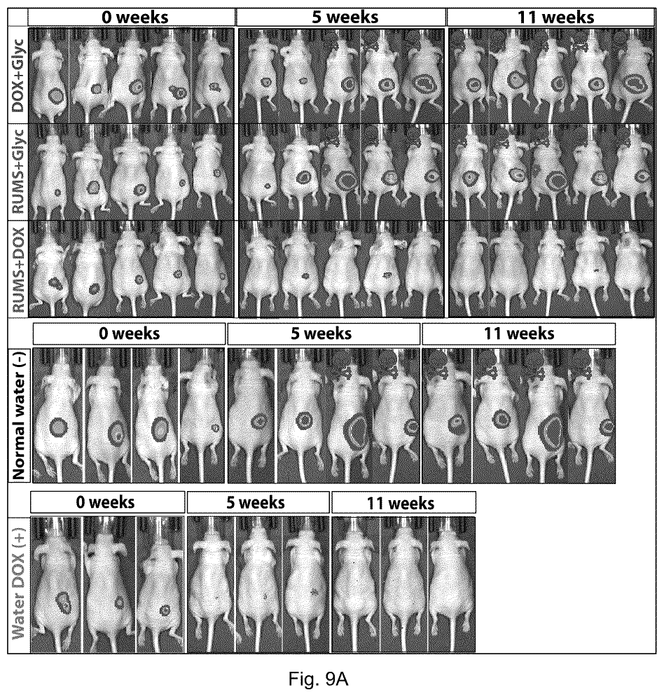

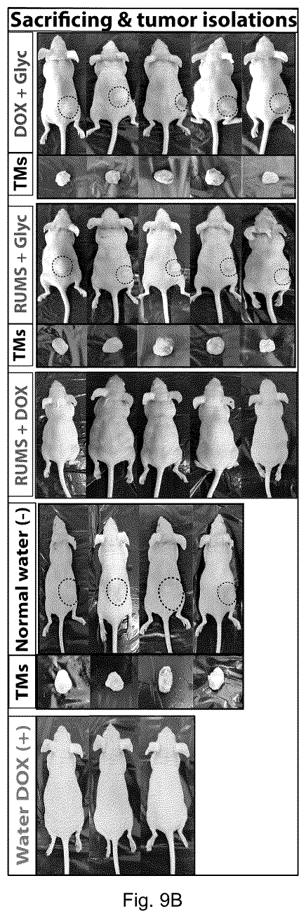

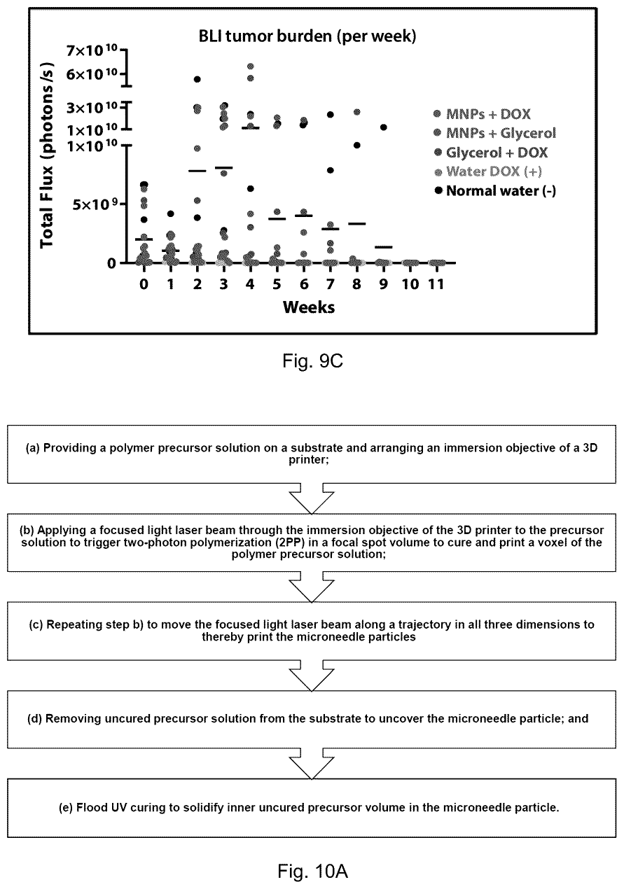

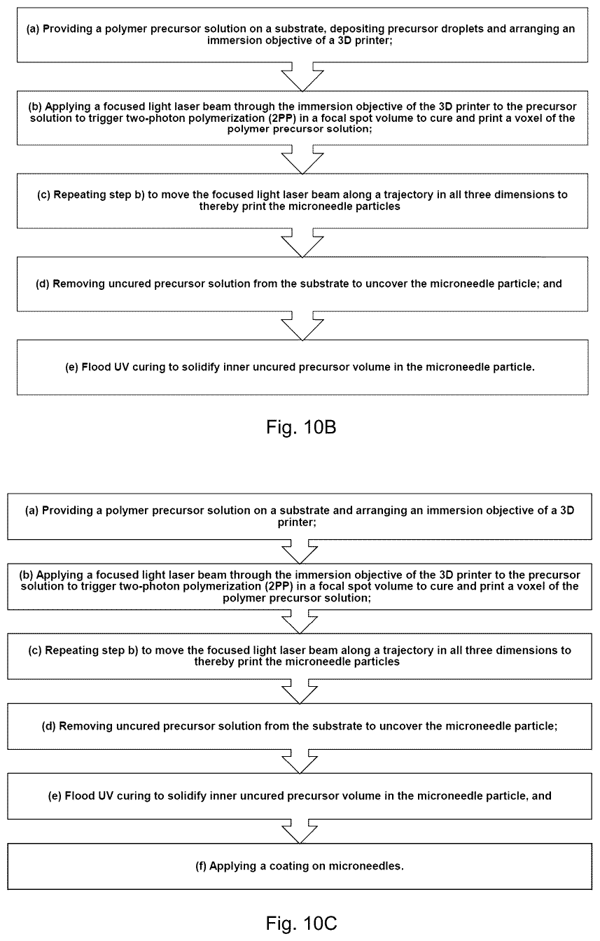

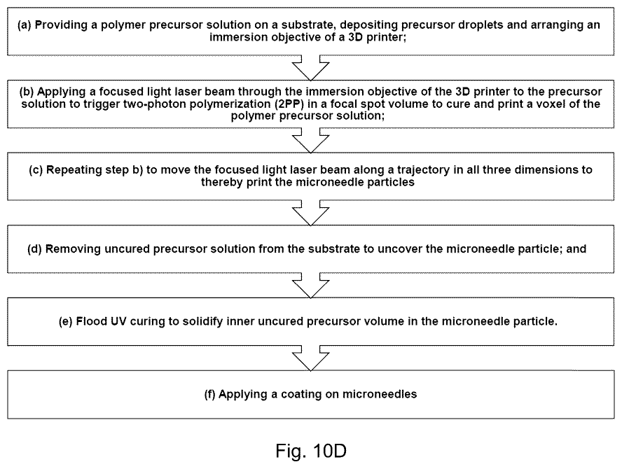

The first step in proving the MNP concept were experiments where the penetration of the SC as a result of the designed rolling motion of the particles could be verified and quantified. Penetration of SC was confirmed by gentian violet (GV) and hematoxylin-eosin (H&E) staining of treated areas on harvested porcine ear skin and murine skin (FIG. 1F, 2A-2L, 2N-2Q). Twenty-five MNP were applied manually with the index finger (˜40 kPa pressure) in a circular motion for 10 s on a 4 cm² area. After standard protocols of staining and washing the excess gentian violet dye, en face brightfield microscopy pictures revealed extensive SC poration with approximately 4500 penetrations via MNP rolling motion (FIGS. 1F, 2B, 2D, 2L). After close-up observation of the application site, the rolling motion of the MNP became evident, as the punctures resembled the pentagon geometry of the needle arrangement (FIG. 1F). The observable “streaks” of this geometric pattern revealed the rolling motion of the MNP during the rubbing application, and their ability to cause multiple penetrations over a large area (FIG. 1F, 2D-2F, 2H, 2J, 2L). Closer microscopy, both en face and as a histology section, also provided insight into the geometry of these punctures, confirming the intended effect of minute disruptions of just the stratum corneum. Especially from H&E section imaging, punctures of the stratum corneum approximately 10-25 μm wide and deep that resemble the needle geometry were observed (FIG. 2O). This observation was also corroborated by scanning electron microscopy (SEM) imaging of the MNP after use (FIGS. 8A-D), which revealed corneocytes remaining on the needles, indicating the successful penetration of SC and the depth the needle was able to reach. Cryo-sectioning preserved the gentian violet staining to reveal the diffusion pattern of the dye at the opening of the SC (FIGS. 2P, 2Q), which could also be seen in brightfield at the highest magnification (FIG. 2E).

### Penetration Efficiency of MNP was Governed by Tip Sharpness

To confirm the hypothesis that the tip sharpness was a remarkably important specification in the design, microneedle particles designed with differing tip diameters (1, 5 and 10 μm) (FIG. 2G, 2I, 2K, 7A-7E), but otherwise identical, were used to penetrate harvested porcine ear skin with the same protocol as described above. The following GV staining (FIGS. 2H, 2J, 2L) was used to count the perforations, manually, and as a percentage of the area stained, automatically. This count showed significant differences due to the tip sharpness of the MNP, and a convincing trend of the increasing penetration performance, in terms of penetrations per particle in a single application, as the tip sharpness improved (FIG. 2M).

Only microneedles with sharpness diameters below 20 μm can render efficient perforations and not cause damage to deeper (viable) layers of the skin. Damage or stimuli to viable layers of the skin will cause a host response.

The results indicated that the sharpness enabled by the two-photon polymerization printing resolution is critical to the MNP's effectiveness. Ultrasharp needle tips provided the capability to penetrate the hard SC with minimal pressure and to limit the skin penetration to only the SC layer, thus eliminating immune responses or other adverse effects and enabling the fast healing of the pores rapidly after the application.

There may be an increase of the effective tip diameter after multiple penetrations, but if the initial sharpness was sufficient the particles may continue to penetrate, increasing the final attainable pore count. Some mechanical deterioration of the needles was observed in SEM micrographs of retrieved MNP after their use (FIG. 8A-8D). As some tips showed some buckling or obscuring by biological material, the effective tip diameter increased a bit; however, some performance was retained, for example on the level seen for the case of 5 μm tip diameter MNP. Solid needles of such size, aspect ratio and mechanical properties as in these MNP were shown to require sharpness specifications that two-photon polymerization could provide.

### MNP Treatment Increased Permeability of Skin to Hydro- and Lipophilic Molecules

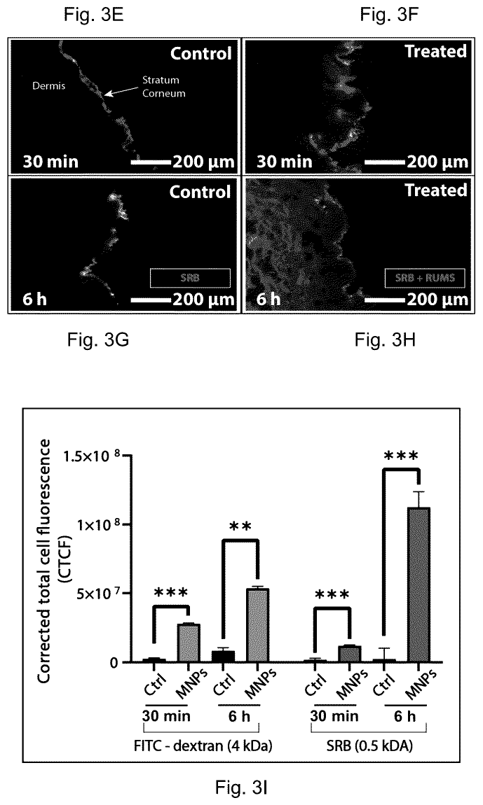

The size and count of pores from the initial ex vivo experiments, demonstrated how MNP could affect the permeability of skin and increase the delivery of compounds that were normally blocked by the SC. Increased skin permeability after MNP treatment was apparent in ex vivo porcine skin experiments where 4 kDa fluorescein isothiocyanate (FITC)-dextran or 0.5 kDa sulforhodamine B (SRB) were topically applied. Increased fluorescent signal of the deeper epidermal layers and the dermis was measured in histological section of samples where MNP treatment took place before applying the respective fluorescent solutions on the skin (FIGS. 3B, 3D, 3F, 3H). In contrast, if no MNP were applied, there was negligible penetration of the compounds deeper into the skin, but saturation on the SC, visualizing its barrier function (FIG. 3 A, C, E, G). The effect was visible at the earlier timepoint of 30 min, and even more pronounced on the concluding timepoint of 6 h after treatment. The data showed a convincing effect of MNP treatment, leading to an increased skin permeability to larger hydro- and lipophilic molecules, quantified as a 7-fold improvement for FITC-dextran (P=0.003) and 70-fold improvement for SRB (P=0.0006) over a 6 h experimental time (FIG. 3I).

### MNP-Aided Topical Doxycycline Treatment Eradicated Subcutaneous Tumors in Murine Flanks

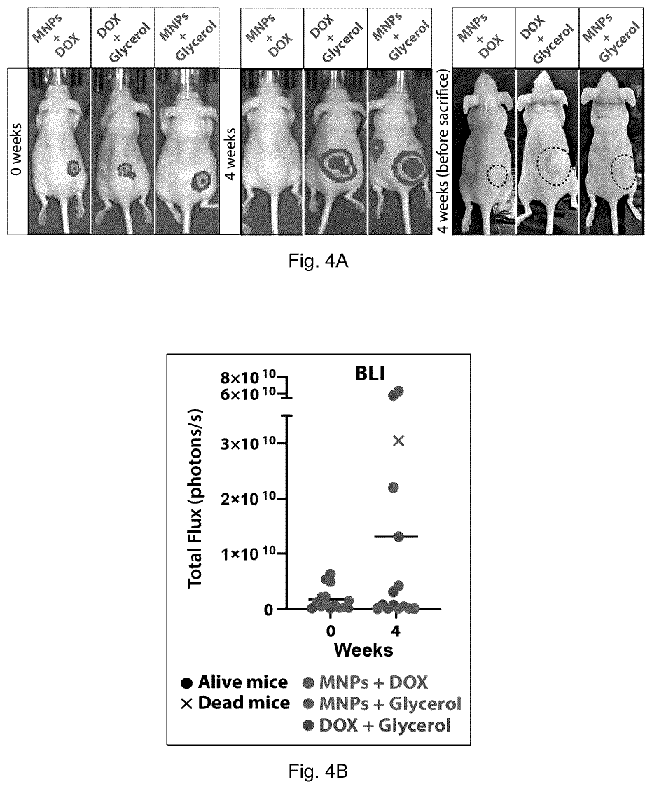

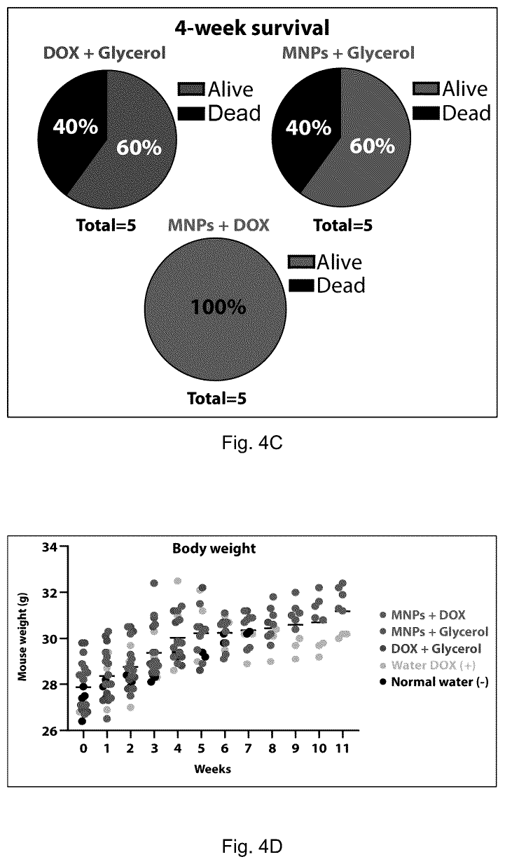

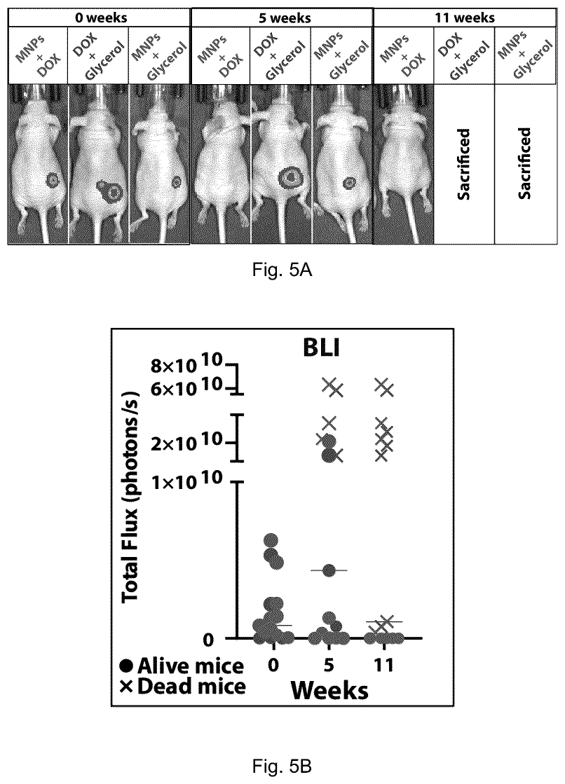

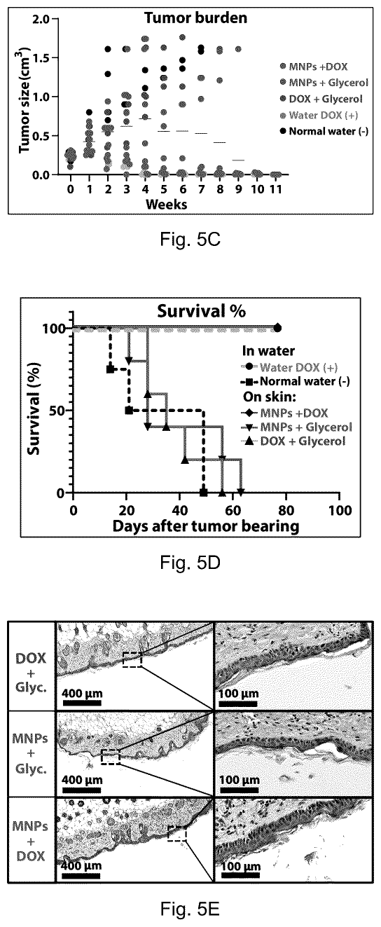

Daily topical skin treatment with MNP and doxycycline+glycerol solution on mice with established flank tumors, from a doxycycline-regulatable Tet-Off brain cancer cell line 40, decreased their size and luciferase signal intensity during the first two weeks of the experiment. Near background levels were reached after 4 weeks of treatment (FIG. 4A-C), with complete recovery observed in a total period of 11 weeks/77 days (FIG. 5 A-C). The mortality rate for the experimental groups of MNP and plain glycerol, and plain doxycycline+glycerol solution was shown to be 40% for both groups after four weeks of treatment, contrasting the 0% mortality of the MNP and doxycycline+glycerol solution group (FIG. 4C). The mean survival time of these two experimental groups was 30 and 35 days, respectively (FIG. 5D). The results of the MNP enhanced doxycycline treatment were more closely correlated with the positive control group undergoing oral systemic administration of doxycycline added in the water supply, rather than the groups receiving topical application (FIG. 4A-4D,5A-5D).

### Daily MNP Treatment Did Not Cause Apparent Adverse Effects in Mice

No differences in the weight of mice were observed among the different MNPMNP skin-treated conditions (FIG. 4D). Doxycycline exposure did not alter the weight of the mice under the period of 11 weeks when it was topically applied, regardless of whether MNP was included (FIG. 4D). Mice under long term oral doxycycline use showed a slower increase in their weight compared to the plain water control group, but no further adverse effects were observed (FIG. 4D). The experimental groups did not exhibit any behavioral signs of discomfort, maintaining normal posture and mobility during the experiment. After the completion of the in vivo trial, the condition of the excised skin was assessed by histological examination which showed no apparent differences among the various groups after 11 weeks of daily treatment (FIG. 5E). No observable skin irritation during treatment and no-cell recruitment on the examined layers of the epidermis was seen by histology (FIG. 5E). Slight dehydration of the skin on the MNP-treated groups was observed regardless of the presence of doxycycline. Histological examination under the microscope showed a normal condition of the epidermal layers among the various groups without any visible damage of the stratum corneum. (FIG. 5E).

### Discussion

The aim of this work was to utilize novel microfabrication techniques and engineering design to open new avenues for transdermal drug delivery in the form of miniature pores in the main barrier layer of the skin, the stratum corneum. This barrier was the main obstacle to hydrophilic and large molecules entering the deeper dermal layers and eventually the circulation ^{1,12}. Therefore, even micron scale pores in the SC increased the delivery of such molecules to an appreciable extent ⁷. The goal was to disrupt this barrier for enhanced drug delivery while not sacrificing safety, tolerability, and ease of use ^{25,37}.

Minimizing the disruption and the created pore size, not only effectively eradicated any pain or even perception of the treatment, but also prevented irritation and infection side effects. Such small pores were expected to heal very quickly, even if occluded by the topical compound at first ³⁵. Therefore, compared to previously reported methods, MNP micro-poration was a better compromise between enabling drug delivery and compromising the shielding properties skin. Larger microneedle applications can cause erythema, irritation, or infection ^{30,41}. They require larger, perceptible tools, such as rollers, pens, or applicators. Minimal disruption of SC is a matter of safety, but also of ease of use and acceptability. In this work, the condition of mice and their skin after daily treatment for 77 days was an indication that minimizing the pores of microneedle treatment was key towards a tolerable transdermal delivery method (FIG. 5E).

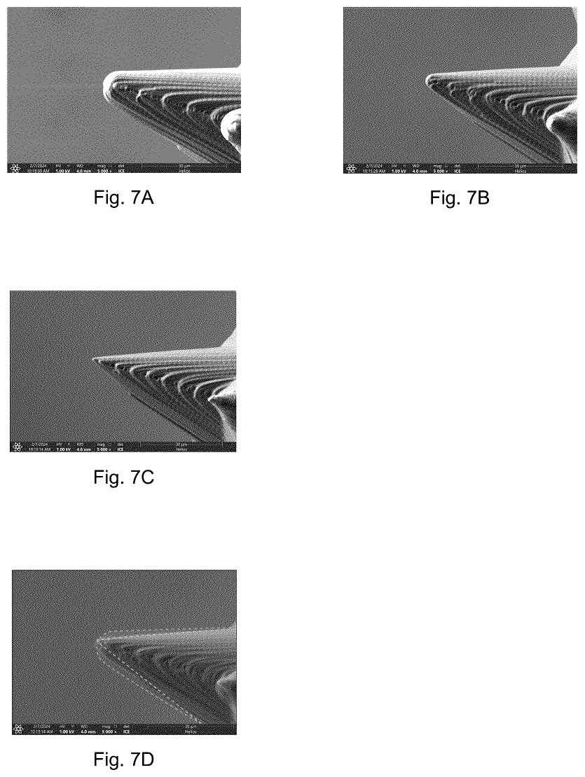

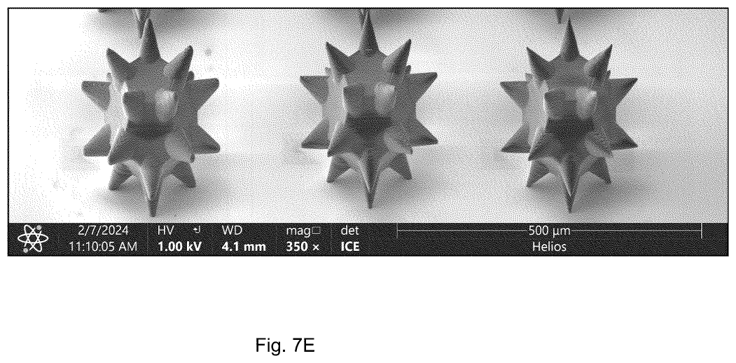

The gentian violet ex vivo staining of porcine and murine skin demonstrated the ability of polymeric, 3D printed, microneedle spheres to achieve thousands of penetrations of the stratum corneum per cm² with a single, simple, topical application (FIGS. 1F, 2). It was shown that engineering design, as in the sharpness of the needles and the geometry enabling the microneedle spheres to roll on the surface of the skin without becoming embedded, was what provides the ability to create multiple pores over a large area of application. The sharpness of the needles on the MNP was shown to be especially important to achieve such a large number of penetrations (FIGS. 2G-2M, 7A-7E). Utilizing softer materials required the needles to be designed with a geometry aspect that made them mechanically stronger (FIG. 1D), as fracture would cause them to be embedded on the patients' skin and would be unacceptable. While some mechanical failure like buckling or occlusion by cellular material or mucus/dirt occurred during use (FIG. 8A-8D), the penetration of the SC at a very low pressure remained. In this work it was shown that the sharpness enabled by the resolution of multi-photon 3D printing provided the capability and safety margins to achieve these requirements and, therefore, efficient multi-poration. The effect of such multi-poration was to increase the permeability of porcine skin to larger, hydrophilic molecules from one to two orders of magnitude (FITC-dextran, SRB) (FIG. 3A-3I).

The design of MNP facilitated topical application, which is the standard practice in dermatology and the most acceptable and intuitive procedure as far as patients are concerned. Compared to microneedle array patches, MNP embedded in a cream can be smeared over square decimeter sized areas, over curved surfaces, around sensitive areas like lips, mouth, nose etc. ²¹. The same can be said for the efficiency of penetration, since the number of pores was not so closely tied to the number of microneedles on the device. Especially self-interference against penetration related to increasing the number of microneedles on a patch was absent (“bed of nails effect”). As a result, the microneedling approach can be expanded further than what microneedle array patches could provide.

In most cases, current dermatological practice cannot completely utilize the advantages of topical applications though, and often resorts to systemic delivery orally, parenterally or by localized injection, with the systemic side effects and the reduced patient tolerability that entails ¹. The inventors'animal study aimed to indicate that MNP-aided increased skin permeability expected by the initial ex vivo experiments can be utilized to deliver compounds topically. The topical transdermal delivery of doxycycline in doxycycline-sensitive subcutaneous tumors in mice was correlated to oral delivery if aided by MNP, or was completely ineffective if not (FIGS. 4, 5). This was considered an important indication that compounds previously unable to be delivered topically would now be enabled to cross the SC into the dermis and subcutaneous space if the formulation contained MNP. The advantages of topical delivery were retained, while the drawbacks of microneedling and the disruption of SC were minimized ^{31,33,36,41}.

In summary, this work indicated that microneedle ultra-miniaturization and intelligent geometrical design, enabled by novel fabrication methods, addressed most of the drawbacks of the state-of-the-art solutions for enhancing transdermal topical delivery. Translation of the MNP technology to clinical use could pave the way for widespread use of biologic, and perhaps personalized, therapeutics that are on the horizon, hand in hand with ease-of-use and tolerability by patients.

### References

1. Dermatology. (elsevier, Philadelphia, Pa., 2018).
2. Hay, R. J. et al. The Global Burden of Skin Disease in 2010: An Analysis of the Prevalence and Impact of Skin Conditions. Journal of Investigative Dermatology 134, 1527-1534 (2014).
3. Lim, H. W. et al. The burden of skin disease in the United States. Journal of the American Academy of Dermatology 76, 958-972.e2 (2017).
4. Cazzaniga, S. et al. Medical, psychological and socio-economic implications of chronic hand eczema: a cross-sectional study. Acad Dermatol Venereol 30, 628-637 (2016).
5. Moustafa, F., Lewallen, R. S. & Feldman, S. R. The psychological impact of rosacea and the influence of current management options. Journal of the American Academy of Dermatology 71, 973-980 (2014).
6. Beyer, V. & Wolverton, S. E. Recent trends in systemic psoriasis treatment costs. Arch Dermatol 146, 46-54 (2010).
7. Prausnitz, M. R. & Langer, R. Transdermal drug delivery. Nat Biotechnol 26, 1261-1268 (2008).
8. Watkinson, A. C., Kearney, M. -C., Quinn, H. L., Courtenay, A. J. & Donnelly, R. F. Future of the transdermal drug delivery market—have we barely touched the surface? Expert Opin Drug Deliv 13, 523-532 (2016).
9. Benson, H. A. E. & Watkinson, A. C. Transdermal and Topical Drug Delivery: Principles and Practice. (2012). doi:10.1002/9781118140505.
10. Pastore, M. N., Kalia, Y. N., Horstmann, M. & Roberts, M. S. Transdermal patches: history, development and pharmacology. Br J Pharmacol 172, 2179-2209 (2015).
11. Choy, Y. B. & Prausnitz, M. R. The rule of five for non-oral routes of drug delivery: ophthalmic, inhalation and transdermal. Pharm Res 28, 943-948 (2011).
12. Sharma, S. & Hani, Y. Anatomy, Skin (Integument), Epidermis. in Anatomy, Skin (Integument), Epidermis (2017).
13. Santaella, R. M. & Fraunfelder, F. W. Ocular Adverse Effects Associated with Systemic Medications. Drugs 67, 75-93 (2007).
14. Alexander, A. et al. Approaches for breaking the barriers of drug permeation through transdermal drug delivery. Journal of Controlled Release 164, 26-40 (2012).
15. Rademaker, M. Adverse effects of isotretinoin: A retrospective review of 1743 patients started on isotretinoin. Australas J Dermatol 51, 248-253 (2010).
16. Wollina, U., Ständer, K. & Barta, U. Toxicity of methotrexate treatment in psoriasis and psoriatic arthritis—short- and long-term toxicity in 104 patients. Clin Rheumatol 20, 406-410 (2001).
17. Wortmann, G. et al. Lipsosomal Amphotericin B for Treatment of Cutaneous Leishmaniasis. The American Society of Tropical Medicine and Hygiene 83, 1028-1033 (2010).
18. Abraham, M. H., Chadha, H. S. & Mitchell, R. C. The Factors that Influence Skin Penetration of Solutes*. Journal of Pharmacy and Pharmacology 47, 8-16 (1995).
19. Karande, P., Jain, A., Ergun, K., Kispersky, V. & Mitragotri, S. Design principles of chemical penetration enhancers for transdermal drug delivery. Proc Natl Acad Sci USA 102, 4688-4693 (2005).
20. Arora, A., Prausnitz, M. & Mitragotri, S. Micro-scale Devices for Transdermal Drug Delivery. Int J Pharm 364, 227-236 (2008).
21. Prausnitz, M. R. Engineering Microneedle Patches for Vaccination and Drug Delivery to Skin. Annu Rev Chem Biomol Eng 8, 177-200 (2017).
22. Ye, Y., Yu, J., Wen, D., Kahkoska, A. R. & Gu, Z. Polymeric microneedles for transdermal protein delivery. Adv Drug Deliv Rev 127, 106-118 (2018).
23. Nguyen, T. T. & Park, J. H. Human studies with microneedles for evaluation of their efficacy and safety. Expert Opinion on Drug Delivery 15, 235-245 (2018).
24. Ripolin, A. et al. Successful application of large microneedle patches by human volunteers. Int J Pharm 521, 92-101 (2017).
25. Roxhed, N., Samel, B., Nordquist, L., Griss, P. & Stemme, G. Painless Drug Delivery Through Microneedle-Based Transdermal Patches Featuring Active Infusion. IEEE Trans. Biomed. Eng. 55, 1063-1071 (2008).
26. Roxhed, N., Gasser, T. C., Griss, P., Holzapfel, G. A. & Stemme, Gö. Penetration-Enhanced Ultrasharp Microneedles and Prediction on Skin Interaction for Efficient Transdermal Drug Delivery. J. Microelectromech. Syst. 16, 1429-1440 (2007).
27. Han, T. & Das, D. B. Potential of combined ultrasound and microneedles for enhanced transdermal drug permeation: A review. European Journal of Pharmaceutics and Biopharmaceutics 89, 312-328 (2015).
28. Park, J. -H., Choi, S. -O., Seo, S., Choy, Y. B. & Prausnitz, M. R. A microneedle roller for transdermal drug delivery. European Journal of Pharmaceutics and Biopharmaceutics 76, 282-289 (2010).
29. Tadros, A. R. et al. STAR particles for enhanced topical drug and vaccine delivery. Nature Medicine 26, 341-347 (2020).
30. Arya, J. et al. Tolerability, usability and acceptability of dissolving microneedle patch administration in human subjects. Biomaterials 128, 1-7 (2017).
31. Bal, S. M., Caussin, J., Pavel, S. & Bouwstra, J. A. In vivo assessment of safety of microneedle arrays in human skin. European Journal of Pharmaceutical Sciences 35, 193-202 (2008).
32. Chen, B. Z. et al. Safety Evaluation of Solid Polymer Microneedles in Human Volunteers at Different Application Sites. ACS Appl. Bio Mater. 2, 5616-5625 (2019).
33. Gill, H. S., Denson, D. D., Burris, B. A. & Prausnitz, M. R. Effect of microneedle design on pain in human volunteers. Clin J Pain 24, 585-594 (2008).
34. Gomaa, Y. A. et al. Effects of microneedle length, density, insertion time and multiple applications on human skin barrier function: assessments by transepidermal water loss. Toxicol In Vitro 24, 1971-1978 (2010).
35. Gupta, J., Gill, H. S., Andrews, S. N. & Prausnitz, M. R. Kinetics of skin resealing after insertion of microneedles in human subjects. Journal of Controlled Release 154, 148-155 (2011).
36. Jeong, H. -R., Lee, H. -S., Choi, I. -J. & Park, J. -H. Considerations in the use of microneedles: pain, convenience, anxiety and safety. Journal of Drug Targeting 25, 29-40 (2017).
37. Kaushik, S. et al. Lack of Pain Associated with Microfabricated Microneedles: Anesthesia and Analgesia 502-504 (2001) doi:10.1097/00000539-200102000-00041.
38 Kim, Y., Jung, J. H., Tadros, A. R. & Prausnitz, M. R. Tolerability, acceptability, and reproducibility of topical STAR particles in human subjects. Bioengineering & Transla Med 8, e10524 (2023).
39. Stampfl, J., Liska, R. & Ovsianikov, A. Multiphoton Lithography: Techniques, Materials, and Applications. (John Wiley & Sons, 2016).
40. Swartling, F. J. et al. Distinct Neural Stem Cell Populations Give Rise to Disparate Brain Tumors in Response to N-MYC. Cancer Cell 21, 601-613 (2012).
41. Alster, T. S. & Graham, P. M. Microneedling: A Review and Practical Guide. Dermatol Surg 44, 397-404 (2018).

1. A microneedle particle arranged to be rolled on a skin of a subject by massaging the particle on the skin of the subject, to thereby penetrate a stratum corneum of the skin, comprising:
a support body, and
a plurality of microneedles protruding from the support body, each microneedle comprising a tip,
wherein the support body maintains the microneedles and the tips in a predetermined conformation which allows the microneedle particle to be rolled on the skin of the subject,
wherein the microneedles have an average tip sharpness radius of less than 20 μm,
wherein the microneedles have an average microneedle length of less than 100 μm, and
wherein the microneedle particle has an average tip sharpness to average microneedle length ratio of 1:5 to 1:200.

2. The microneedle particle according to claim 1, wherein the support body maintains the microneedles and the tips in a predetermined conformation in which the tips of the microneedles define a 3D convex hull configured to be rolled on the skin, wherein the 3D convex hull has a shape of a cylindroid, a cylinder, a spheroid, a sphere, a polyhedron, an oloid, a Steinmetz solid, or a Gomboc shape.

3. The microneedle particle according to claim 1, wherein the microneedle length is 10-50 μm, preferably not more than 40 μm.

4. The microneedle particle according to claim 1, comprising between 10 and 40 microneedles.

5. The microneedle particle according to claim 1, wherein a distance between two adjacent microneedle tips is between 10 μm and 100 μm.

6. The microneedle particle according to claim 1, comprising a formulation of at least 0.004% wt concentration of microneedle particles.

7. The microneedle particle according to claim 1, wherein the microneedle particle comprises a biocompatible material.

8. The microneedle particle according to claim 1, wherein the microneedle particle is made of a monolithic material.

9. The microneedle particle according to claim 1, wherein the microneedle particle is made of a material having a Young's modulus of more than 0.5 giga Pascal (GPa).

10. The microneedle particle according to claim 1, wherein the microneedle particle has a largest dimension of up to about 500 μm.

11. The microneedle particle according to claim 10, wherein the microneedle particle has a largest dimension of more than 40 μm.

12. The microneedle particle according to claim 1, wherein a volume of a single particle is from 6.5e-5 mm³ to 2 mm³.

13. The microneedle particle according to claim 1, wherein the microneedles comprise a coating, wherein the coating is selected from at least one of a ceramic layer, a carbon coating, and an ALO deposition.

14. The microneedle particle according to claim 1, wherein the support body is essentially a sphere or essentially a polyhedron.

15. The microneedle particle according to claim 1, wherein the largest dimension of the support body is 10-500 μm.

16. The microneedle particle according to claim 1, having a weight of approximately 0.5 to 30 μg.

17. A formulation comprising a plurality of microneedle particles according to claim 1, and a non-solid medium in which the microneedle particles are dispersed, wherein the formulation is adapted for application to a skin of a mammal.

18. A formulation comprising a plurality of microneedle particles and a non-solid medium in which the microneedle particles are dispersed, wherein the microneedle particle is arranged to be rolled on a skin of a subject by massaging the particle on the skin of the subject, to thereby penetrate a stratum corneum of the skin, the microneedle particle comprising:
a support body, and
a plurality of microneedles protruding from the support body, each microneedle comprising a tip,
wherein the support body maintains the microneedles and the tips in a predetermined conformation which allows the microneedle particle to be rolled on the skin of the subject,
and wherein the concentration of the microneedle particles in the formulation is 0.01 % wt or less.

19. The formulation comprising a plurality of microneedle particles according to claim 17, wherein the non-solid medium comprises at least one of a gel, a serum, a cream, a lotion, a cosmetic cream, a cosmetic fluid, and a medicament.

20. The formulation comprising a plurality of microneedle particles according to claim 18, wherein the non-solid medium comprises at least one of a gel, a serum, a cream, a lotion, a cosmetic cream, a cosmetic fluid, and a medicament.
